(19) United States
(12) Patent Application Publication
(10) Pub. No.: US 2026/0146297 A1
(43) Pub. Date: May 28, 2026

(54) METAGENOMIC DATA FILTERING FOR HEALTH DIAGNOSTICS, FOOD QUALITY AND SAFETY, AND SURROUNDING ENVIRONMENTAL SAFETY

(71) Applicant: Mars, Incorporated, McLean, VA (US)

(72) Inventors: Balasubramanian GANESAN, Vancouver, WA (US); Robert C. BAKER, Geylang (SG); David F. CREAN, Harringworth (GB); Bart C. WEIMER, Davis, CA (US)

(73) Assignee: Mars, Incorporated, McLean, VA (US)

(21) Appl. No.: 19/133,644

(22) PCT Filed: Nov. 27, 2023
(86) PCT No.: PCT/US2023/081106

§ 371 Date: May 28, 2025

Related U.S. Application Data
(60) Provisional application No. 63/429,391, filed on Dec. 1, 2022.

(51) Int. Cl. C12Q 1/689; C12Q 1/6874; C12Q 1/70

(52) U.S. Cl. CPC C12Q 1/689; C12Q 1/6874; C12Q 1/70

(57) ABSTRACT

Methods and systems for detecting unwanted microbes, and the origins thereof, in an environment are disclosed herein. The methods disclosed herein can be applied to a wide range of environments, including, but not limited to, food production facilities, office facilities, and public facilities. The methods comprise obtaining one or more samples from one or more locations in an environment, sequencing a plurality of nucleic acid sequences within the samples, identifying one or more indicator microbial signatures or origin-indicating microbial signatures within the plurality of nucleic acid sequences, comparing the indicator microbial signatures against one or more databases, and identifying one or more unwanted microbes, and/or the origins thereof, that correspond to one or more of the microbial signatures identified.

### CROSS-REFERENCE TO RELATED APPLICATIONS

This application claims the benefit of U.S. Provisional Application No. 63/429,391 filed Dec. 1, 2022, the entire contents which are incorporated herein by reference.

### FIELD OF THE DISCLOSURE

This disclosure relates to methods and systems for detecting unwanted microbes and origins thereof in an environment using microbiome analysis and metagenomic data filtering.

### BACKGROUND OF THE DISCLOSURE

Infectious disease and food safety are of particular importance for public health management. Efficient and reliable techniques for determining the presence of unwanted microbes in human environments, including in food production environments, are needed. Existing methods have relied on targeted detection of pathogens and other contaminants by chemical or biological means, such as through qPCR, chromatography, or enzymatic analysis. High throughput methods for analysis of nucleic acids or proteins from environmental samples for direct identification of sequences from pathogenic or contaminating microbes, for example, using sequencing or proteomics, have also been developed. However, these methods require that the pathogenic or contaminating microbe is present at a certain minimum abundance to be detected, which results in some unwanted microbes going undetected. Further, existing methods are not designed to trace the unwanted microbe back to its origin, i.e., its point of introduction into the environment.

Accordingly, there is a need for high-throughput end-to-end methods for detecting unwanted microbes in human environments, including in food production environments. Moreover, there is a need for methods of tracing unwanted microbes back to their contamination events to remediate or prevent hazards to human or animal health.

### SUMMARY OF THE INVENTION

Disclosed herein are systems and methods for detecting one or more unwanted microbes in an environment, and for determining the origin or origins of the unwanted microbes. The methods and systems can be used for identifying unwanted microbes in a wide range of environments including, but not limited to, clinical, food production and distribution environments. The methods and systems disclosed herein are based on the analysis of microbial signatures corresponding to one or more unwanted microbes present in a sample (i.e., indicator microbial signatures) and microbial signatures corresponding to one or more origins of an unwanted microbe (i.e., origin-indicating microbial signatures). As for example, food products contaminated with unwanted microbes contain different indicator and origin-indicating microbial signatures that can be detecting by sequencing nucleic acids in environmental samples. Because these methods use metagenomic data analysis to identify microbial signatures, they can be used to detect unwanted microbes that are present below the limit of detection of existing methods, and to trace unwanted microbes back to their origin, i.e., the point of introduction of the unwanted microbe into the environment being analyzed. This allows the user to precisely pinpoint environmental health and safety issues and make decisions to correct or prevent the introduction of unwanted microbes into the environment.

In one aspect, disclosed herein is a method for detecting one or more unwanted microbes in an environment, the method comprising: obtaining one or more samples from one or more locations associated with the one or more unwanted microbes in the environment; sequencing a plurality of nucleic acid sequences within the one or more samples; identifying one or more indicator microbial signatures within the plurality of nucleic acid sequences; comparing the one or more identified indicator microbial signatures against one or more databases of indicator microbial signatures that correspond to particular unwanted microbes; and identifying one or more unwanted microbes that correspond to one or more of the identified indicator microbial signatures based on the comparison. In some embodiments, the one or more indicator microbial signatures comprise nucleic acid sequences from microbes other than the one or more unwanted microbes. In certain embodiments, the one or more indicator microbial signatures do not comprise nucleic acid sequences from the one or more unwanted microbes.

In another aspect, disclosed herein is a method for identifying one or more origins of one or more unwanted microbes in an environment, the method comprising: obtaining one or more samples from one or more locations associated with the one or more unwanted microbes in the environment; sequencing a plurality of nucleic acid sequences within the one or more samples; identifying one or more origin-indicating microbial signatures within the plurality of nucleic acids sequences; comparing the one or more identified origin-indicating microbial signatures against one or more databases of origin-indicating microbial signatures that correspond to particular origins of unwanted microbes; and identifying one or more origins of the one or more unwanted microbes that correspond to one or more of the identified origin-indicating microbial signatures based on the comparison. In some embodiments, the one or more origin-indicating microbial signatures comprise nucleic acid sequences from microbes other than the one or more unwanted microbes. In certain embodiments, the one or more origin-indicating microbial signatures do not comprise nucleic acid sequences from the one or more unwanted microbes.

In some embodiments, the method for identifying one or more origins of one or more unwanted microbes in an environment further comprises obtaining one or more origin samples from one or more potential origins of the one or more unwanted microbes; sequencing a plurality of origin nucleic acid sequences within the one or more origin samples; detecting one or more nucleic acid sequences of the one or more unwanted microbes in one or more of the origin samples; and identifying one or more origins of the one or more unwanted microbes based on detecting the one or more nucleic acid sequences of the one or more unwanted microbes in one or more of the origin samples. In certain embodiments, the method further comprises identifying an unknown origin-indicating microbial signature within the plurality of origin nucleic acids sequences of the one or more origin samples in which the one or more unwanted microbes were detected. In certain embodiments, the method further comprises adding the unknown origin-indicating microbial signature and the corresponding origin of unwanted microbes to the one or more databases. In some variations, the method comprises obtaining two, three, four, five, ten, or more origin samples from two, three, four, five, ten, or more potential origins of the one or more unwanted microbes.

In some embodiments of the method for identifying one or more origins of one or more unwanted microbes in an environment, the one or more origin-indicating microbial signatures correspond to one or more unwanted microbes associated with a single origin. In certain embodiments, the one or more origin-indicating microbial signatures correspond to one or more microbes associated with two or more origins. In certain embodiments, the one or more origins of the one or more unwanted microbes comprise an animal or livestock origin. In some variations, the livestock origin is dairy, egg, poultry meal, fish meal, bone meal, a bovine meat, an ovine meat, turkey, chicken, duck, or goose. In additional variations, the animal origin is a companion animal or a service animal (e.g., a dog or a cat). In certain embodiments, the one or more origins of the one or more unwanted microbes comprise a plant origin or a fungal origin. In some variations, the plant origin is rice, wheat, maize, chickpeas, lima beans, peanuts, kidney beans, cashews, walnuts, pecans, or hazel nuts. In certain embodiments, the one or more origins of the one or more unwanted microbes comprise an agricultural or farm soil, an agricultural or farm equipment surface, a crop, a livestock animal, a transport vehicle surface, a factory equipment surface, a factory floor, a food processing material, a food processing equipment surface, a food product, a worker apparel, or any combination thereof. In certain embodiments, the one or more origins of the one or more unwanted microbes comprise an origin that corresponds to a particular geographical region. In other embodiments, the one or more origins comprise clinical samples from human or other animals or clinical equipment and environments.

In some embodiments of the preceding methods, two, three, four, five, ten, or more samples are collected from two three, four, five, ten, or more locations within the environment. In additional embodiments of the preceding methods, two, three, four, five, ten, or more samples are collected from two, three, four, five, ten, or more environments.

In some embodiments of the preceding methods, the environment is a food production environment. In certain embodiments, the food production environment is selected from the group consisting of a farm, a food transport vehicle, an animal transport vehicle, a food processing facility, a food packaging facility, a food distribution facility, a warehouse, and a food market. In one variation, the food production environment is an animal transport vehicle. In certain embodiments, two, three, four, five, ten, or more samples are collected from two, three, four, five, ten, or more environments in a food production chain. In certain embodiments, the one or more samples comprise samples from agricultural or farm soil, agricultural or farm equipment, crops, livestock, transport vehicle surfaces, factory equipment, factory floors, food processing material, food processing equipment, food products, worker apparel, or any combination thereof. In other embodiments, the one or more origins comprise clinical samples from human or other animals or clinical equipment and environments.

In other embodiments of the preceding methods, the environment is an environment in a clinic, public building, an office building, a residential building, or an animal care facility. In certain embodiments, the environment is an office or a restroom. In certain embodiments, the one or more samples comprise samples from a companion animal, a service animal, an item or surface contacted by a companion animal or a service animal, or any combination thereof.

In other embodiments of the preceding methods, the environment is a spray dryer. Accordingly, in one aspect, disclosed herein is a method for detecting one or more unwanted microbes in a spray dryer, the method comprising: obtaining one or more samples from one or more locations within the spray dryer, from a powder produced by the spray dryer, or from a liquid to be fed into the spray dryer, or any combination thereof in the spray dryer, around the spray dryer, or both; sequencing a plurality of nucleic acid sequences within the one or more samples; identifying one or more indicator microbial signatures within the plurality of nucleic acid sequences; comparing the one or more identified indicator microbial signatures against one or more databases of indicator microbial signatures that correspond to particular unwanted microbes; and identifying one or more unwanted microbes that correspond to one or more of the identified indicator microbial signatures based on the comparison. In some embodiments, the one or more indicator microbial signatures comprise nucleic acid sequences from microbes other than the one or more unwanted microbes. In certain embodiments, the one or more indicator microbial signatures do not comprise nucleic acid sequences from the one or more unwanted microbes.

In some embodiments of the method for detecting one or more unwanted microbes in a spray dryer, two, three, four, five, ten, or more samples are collected from two, three, four, five, ten, or more locations within the spray dryer or on the surfaces of the spray dryer. In certain embodiments, the one or more samples comprise one or more samples from the inlet, the outlet, the storage silos, the drying chamber, or the cyclone of the spray dryer, or any combination thereof. In some variations, the liquid to be fed into the spray dryer is milk. In some additional variations, the powder produced by the spray dryer is milk powder.

In some embodiments of any of the foregoing methods, the one or more indicator microbial signatures or origin-indicating microbial signatures correspond to the genus taxonomy of one or more unwanted microbes present in the environment. In other embodiments of any of the foregoing methods, the one or more indicator microbial signatures or origin-indicating microbial signatures correspond to the species or serotype taxonomy of one or more unwanted microbes present in the environment. In some embodiments of any of the foregoing methods, the one or more indicator microbial signatures or origin-indicating microbial signatures correspond to the relative abundance of the one or more unwanted microbes present in the environment.

In some embodiments of any of the foregoing methods, the one or more unwanted microbes are selected from the group consisting of bacteria, viruses, archaea, and eukaryotic microorganisms. In certain embodiments of any of the foregoing methods, the one or more unwanted microbes belong to a genus taxonomy selected from the group consisting of: Parageobacillus, Blautia, Aliivibrio, Porphyrobacter, Shigella, Aneurinibacillus, Anaerostipes, Photobacterium, Erythrobacter, Rathayibacter, Butyrivibrio, Tyzzerella, Grimontia, Dechloromonas, Leifsonia, Coprothermobacter, Intestinimonas, Pseudoalteromonas, Pseudarthrobacter, Arthrobacter, Megasphaera, Ethanoligenens, Alteromonas, Isoptericola, Micrococcus, Eubacterium, Colwellia, Cellulomonas, Thermus, Oscillibacter, Yersinia, Nocardia, Meiothermus, Weissella, Edwardsiella, Gordonia, Rahnella, Murdochiella, Oceanimonas, Propionibacterium, Azotobacter, Eggerthella, Marinomonas, Tessaracoccus, Caulobacter, Adlercreutzia, Halomonas, Pimelobacter, Fibrobacter, Gordonibacter, Methylophaga, Actinoplanes, Fervidobacterium, Obesumbacterium, Brucella, Listeria, Methanobrevibacter, Plesiomonas, Caldanaerobacter, Deinococcus, Methanosarcina, Gallibacterium, Synechococcus, Spirosoma, Thioploca, Calothrix, Helicobacter, Thermotoga, Janthinobacterium, Nonlabens, Barnesiella, Fusobacterium, Ornithobacterium, Ilyobacter, Akkermansia, Thermodesulfobacterium, Cloacibacillus, Theileria, Gyrovirus, T7virus, T4virus, Alpharetrovirus, Sp18virus, Acidaminococcus, Altererythrobacter, Comamonas, Arcobacter, Aeromicrobium, Pediococcus, Proteus, Alistipes, Azospira, Geobacillus, Geoalkalibacter, Agrobacterium, Vibrio, Christensenella, Bosea, Kurthia, Hafnia, Alcaligenes, Clostridioides, Novosphingobium, Oblitimonas, Morganella, Amycolatopsis, Odoribacter, Pseudoxanthomonas, Negativicoccus, Aureimonas, Olsenella, Psychrobacter, Paenibacillus, Brachybacterium, Parabacteroides, Shewanella, Providencia, Brevibacterium, Roseburia, Candida, Ruminococcus, Caulimovirus, Selenomonas, Clavibacter, Treponema, Curtobacterium, Turicibacter, Erwinia, Frondihabitans, Hymenobacter, Kineococcus, Kluyveromyces, Massilia, Methylobacterium, Microbacterium, Nocardioides, Ochrobactrum, Pseudonocardia, Rhizobium, Saccharopolyspora, Sanguibacter, Shinella, Sphingobacterium, Sugiyamaella, Chryseobacterium, Aeromonas, Achromobacter, Blastomonas, Pantoea, Delftia, Anoxybacillus, Bordetella, Mycobacterium, Bacteroides, Brevundimonas, Rhodococcus, Bifidobacterium, Kosakonia, Streptomyces, Desulfovibrio, Sphingobium, Thermothelomyces, Flavonfractor, Sphingomonas, Thielavia, Lachnoclostridium, Sphingopyxis, Macrococcus, Cupriavidus, Moraxella, Prevotella, Ruminiclostridium, Bradyrhizobium, Campylobacter, Clostridium, Stenotrophomonas, Burkholderia, Cutibacterium, Xanthomonas, Serratia, Escherichia, Staphylococcus, Streptococcus, Variovorax, Acidovorax, Acinetobacter, Bacillus, Citrobacter, Corynebacterium, Enterobacter, Enterococcus, Klebsiella, Lactobacillus, Lactococcus, Pseudomonas, Raoultella, and Salmonella.

In some embodiments of any of the foregoing methods, sequencing the plurality of nucleic acid sequences within the one or more samples comprises preparing a sequencing library. In certain embodiments of any of the foregoing methods, sequencing the plurality of nucleic acid sequences within the one or more samples comprises next generation sequencing or microarray analysis. In certain embodiments of any of the foregoing methods, sequencing the plurality of nucleic acid sequences within the one or more samples comprises preparing a sequencing library. In certain embodiments of any of the foregoing methods, the plurality of nucleic acid sequences comprise DNA sequences, RNA sequences, or a combination thereof. In certain embodiments of any of the foregoing methods, non-microbial sequences are filtered from the plurality of nucleic acid sequences prior to identifying the one or more indicator microbial signatures or origin-indicating microbial signatures. In certain embodiments of any of the foregoing methods, the one or more databases are databases of microbial nucleic acid sequences.

In another aspect, disclosed herein is a system for detecting one or more unwanted microbes in an environment, comprising: one or more processors; and a memory comprising instructions executable by the one or more processors that, when executed by the one or more processors, cause the system to: identify one or more indicator microbial signatures within a plurality of nucleic acid sequences obtained from sequencing a plurality of nucleic acid sequences from one or more samples from one or more locations associated with the one or more unwanted microbes in the environment; compare the one or more identified indicator microbial signatures against one or more databases of indicator microbial signatures that correspond to particular unwanted microbes; and identify one or more unwanted microbes that correspond to one or more of the identified indicator microbial signatures based on the comparison.

In yet another aspect, disclosed herein is a system for identifying one or more origins of one or more unwanted microbes in an environment, comprising: one or more processors; and a memory comprising instructions executable by the one or more processors that, when executed by the one or more processors, cause the system to: identify one or more origin-indicating microbial signatures within a plurality of nucleic acids sequences obtained from sequencing a plurality of nucleic acid sequences from one or more samples from one or more locations associated with the one or more unwanted microbes in the environment; compare the one or more identified origin-indicating microbial signatures against one or more databases of origin-indicating microbial signatures that correspond to particular origins of unwanted microbes; and identify one or more origins of the one or more unwanted microbes that correspond to one or more of the identified origin-indicating microbial signatures based on the comparison.

In still another aspect, disclosed herein is a system for detecting an unwanted microbe in spray dryer, comprising: one or more processors; and a memory comprising instructions executable by the one or more processors that, when executed by the one or more processors, cause the system to: identify one or more indicator microbial signatures within a plurality of nucleic acid sequences obtained from sequencing a plurality of nucleic acid sequences from one or more samples from one or more locations within the spray dryer, a powder produced by the spray dryer, a liquid to be fed into the spray dryer, or any combination thereof associated with the one or more unwanted microbes in the spray dryer; compare the one or more identified indicator microbial signatures against one or more databases of indicator microbial signatures that correspond to particular unwanted microbes; and identify one or more unwanted microbes that correspond to one or more of the identified indicator microbial signatures based on the comparison.

In some embodiments of the foregoing systems, the one or more indicator microbial signatures or origin-indicating microbial signatures comprise nucleic acid sequences from microbes other than the one or more unwanted microbes. In certain embodiments of the foregoing systems, the one or more indicator microbial signatures or origin-indicating microbial signatures do not comprise nucleic acid sequences from the one or more unwanted microbes.

### BRIEF DESCRIPTION OF THE FIGURES

FIG. 1 is a flow diagram depicting a method of identifying the origin of an unwanted microbe present in a food production environment.

FIG. 2 is a flow diagram depicting an exemplary data analysis process for the identification of an origin-identifying microbial signature in a sample from a food production environment. The dashed line indicates that microbial identification can be performed directly using a database of nucleic acid sequences corresponding to microbes associated with a particular origin, such as the bacterial species shown in Tables 1-5, to confirm the origin of the unwanted microbe without fully identifying the microbial species present in the sample.

FIG. 3 is an exemplary decision tree of species determination of a single bacterial species, showing how traceback of the origin of an unwanted microbe is achieved based on the presence of the single bacterial species.

FIG. 4 is a decision tree for a working example of determination of the origin of an unwanted microbe based on the presence of a single bacterial species in the sample.

FIG. 5 is an exemplary decision tree of species determination showing how traceback is achieved with multiple bacterial species across multiple possible origins of unwanted microbes. Multiple species together can form an origin-indicating microbial signature. The same species are not considered in each stage, as the species among the g collection are distinct from g−1, g−2, and so on.

FIG. 6 is a decision tree for a working example of species determination showing how traceback is achieved with multiple bacterial species across multiple possible origins of unwanted microbes. Multiple microbial species together can form an origin-indicating microbial signature. The same species are not considered in each stage. Instead of iterative reduction, a combined signature of presence/absence or relative levels of species can also be applied with the same process. Tables 1-5 show examples of databases of bacteria found in various origin materials, either singly or in combination.

FIG. 7 is a flow diagram depicting how the methods described herein can be used in various applications in food quality and safety, public health, and environmental safety.

### DETAILED DESCRIPTION

The following description sets forth exemplary methods, conditions, and the like and are not intended as limiting the scope of the present disclosure. Instead, it is provided as a description of exemplary embodiments.

### I. Overview

Disclosed herein is a microbial origin end-to-end traceback system for multiple purposes that support human and animal health diagnostics, food safety, and environmental safety. This system uses metagenomic microbiome data from any sample material to identify the microbial components within the sample, by using data from high throughput sequencing of nucleic acids. The system can also use microbiome sequence data from smaller scale surveys such as with microarrays or other lower throughput sequencing or PCR and any such data collated from multiple separate assays using nucleic acid. In all cases, as described further below, the invention is based on using metagenomic microbiome data to identify microbial signatures (i.e., indicator microbial signatures and/or origin-indicating microbial signatures) as indicators of existing infection, incoming infection, or unwanted bacteria in food that may cause infection or food spoilage. The microbial signatures can represent a subset of the metagenomics data obtained for a particular sample. The microbial signatures can be determined in a targeted manner by analyzing the data to identify a particular set of microbes associated with specific unwanted microbes and possible origins thereof. Alternatively, the sequence data can be analyzed in an unbiased manner to identify microbial signatures corresponding to detected deviations in the microbiome composition that can then be traced to particular origins of unwanted microbes. This can be applied to animal and human health diagnostics and food product or supply chain safety. The approach can use either one or more targeted analyses of known microbes unique to selective origins, or can also analyze the entirety of the data to identify specific deviations that can then be traced to the point of entry of these unwanted microbes. The microbial compositions are filtered to identify uniquely present bacteria that are then used to forensically trace back to specific origins, e.g., specific food matrix materials or points of contamination, to determine the origin of the microbial species accurately and confirm its point of entry into the food product supply chain.

The methods and systems described herein solve an important problem in monitoring the food product or infectious disease transmission or environmental safety. Most current nucleic acid-based methods for identifying unwanted microbes in the environment and/or in food products rely on direct analysis of sequences encoded by the unwanted microbe, which can fail to detect the microbe if it is present in an amount below the limit of detection. Such methods are also unable to trace back an unwanted microbe to its point of origin in the environment or the food product supply chain. The present disclosure is based at least in part on the discovery that such microbes present in low amounts may be detected by shifting patterns of other associated microbes that either rise or fall in levels in response to the presence of the unwanted microbe. Thus, the methods and systems described herein can identify indicator microbial signatures or origin-indicating microbial signatures that do not comprise the nucleic acid sequences encoded by the unwanted microbe itself but can still indicate its presence in the sample or its origin in the environment or in the food supply chain.

The methods and systems can also be used to test various environments, e.g., the farm soil or manure, clinical surfaces, equipment, or sheets, food facility drains, offices, residences, for sources of microbial incidence or indicators of incoming unwanted bacteria. This can be applied to build consumer confidence in food quality and also to infectious disease and environmental safety diagnostics. The methods and systems described herein can also serve independently as a diagnostic tool for identification of microbes from human and domestic animal (e.g., livestock or companion animal) health testing, such as from feces or urine or other biosamples, to derive indicator microbial signatures of unwanted bacteria. Once an unwanted microbe is traced back to its origin in the environment (e.g., a point of contamination), corrective action can be taken to prevent further contamination. Importantly, the time taken to resolve these issues is greatly shortened by the methods described herein, leading to a determination of any microbial incidence with much improved accuracy and reliability of the testing process.

Although the following description uses terms first, second, etc., to describe various elements, these elements should not be limited by the terms. These terms are only used to distinguish one element from another.

The terminology used in the description of the various embodiments described herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, rational numbers, steps, operations, elements, and/or components, but do not preclude the presence or addition of one or more other features, integers, rational numbers, steps, operations, elements, components, and/or groups thereof.

The term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

The terms “microbiome” or “microbiota” may be used with meanings and/or intention in the art, but as used in this specification, these terms may be construed to encompass any meaning and/or intention used by those in the art, unless otherwise specified.

Metagenomics generally relates to the study of genetic material that is obtained from an environment (e.g., a food production environment), an ecosystem, or a sample therefrom, and allows for analysis of a sample without the need to isolate the genetic material from individual species present in the sample. Metagenomics allows environmental samples to be analyzed in an unbiased, high throughput, and comprehensive manner.

A “microbiome” or “microbiota” generally relates to a community of microbes present in an environment. Shifts in the microbiome composition of a sample from an environment (e.g., in a food product sample or other sample from a food production environment) can reflect a contamination event in the environment. For example, contamination of a meat processing facility with livestock feces can lead to a change in the microbiome of the meat or of meat processing equipment in the facility. Thus, analysis of the microbiome in a particular sample (e.g., a food sample) can be employed to indirectly identify unwanted microbes and origins thereof in an environment (e.g., a food product, a food processing facility, or food processing equipment).

As used herein, “indicator microbial signature” generally refers to a collection of microbial sequences obtained from an environmental sample that indicates the presence of a particular unwanted microbe in the environment from which the sample was obtained. As used herein, “origin-indicating microbial signature” generally refers to a collection of microbial sequences obtained from an environmental sample that indicates the origin of a particular unwanted microbe present in environment from which the sample was obtained. The methods described herein comprise identifying one or more indicator microbial signatures, or one or more origin-indicating microbial signatures, in a plurality of nucleic acid sequences obtained from an environmental sample. The one or more indicator microbial signatures, or one or more origin-indicating microbial signatures, may correspond to all microbes present in the sample, or to a partial list of the microbes present in the sample. For example, the one or more indicator microbial signatures, or one or more origin-indicating microbial signatures, may correspond to only one microbe present in the sample, or may correspond to a subset of multiple microbes present in the sample. Importantly, the indicator microbial signature or origin-indicating microbial signatures may correspond to one or more microbes present in the sample that do not include the unwanted microbe. As used herein, an indicator microbial signature or an origin-indicating microbial signature “corresponds to” a given microbe or microbes present in the sample when the indicator microbial signature includes sequences of the given microbe or microbes. By contrast, an indicator microbial signature can “indicate” the presence of an unwanted microbe even if it does not include sequences of the unwanted microbe, and an origin-indicating microbial signature can “indicate” the origin of the unwanted microbe even if it does not include sequences of the unwanted microbe.

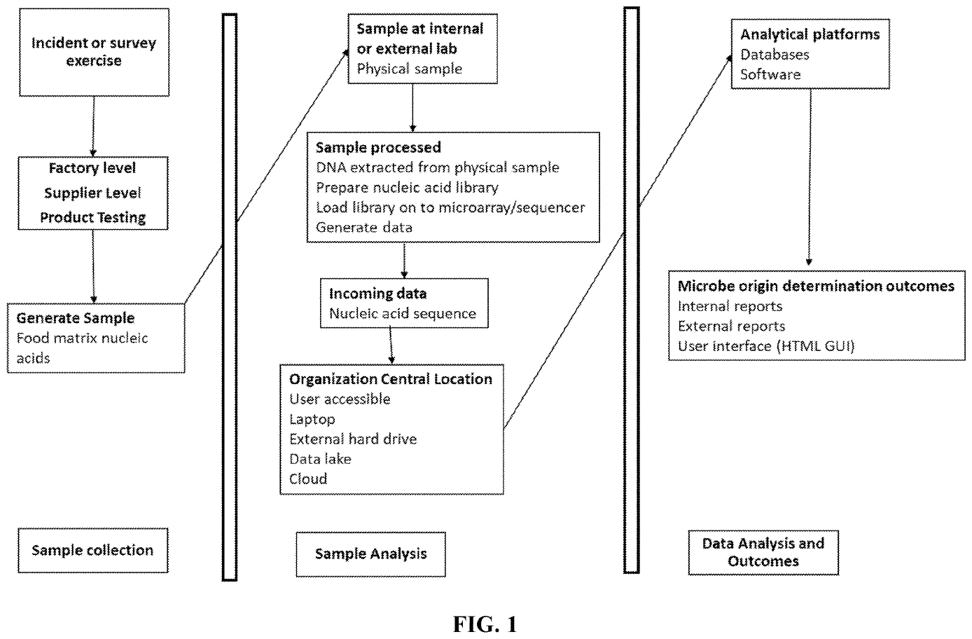

FIGS. 1-7 provide exemplary embodiments of methods for identifying one or more unwanted microbes, and one or more origins thereof, in an environment, wherein the methods comprise obtaining one or more samples from one or more locations associated with the one or more unwanted microbes in the environment, sequencing a plurality of nucleic acid sequences within the one or more samples, and identifying one or more indicator microbial signatures or origin-indicating microbial signatures within the plurality of nucleic acid sequences.

FIG. 1 depicts a flowchart of an exemplary method 100 for identifying the origin of an unwanted microbe in a food production environment. At 102, the process may be initiated in response to an incident or survey exercise. The incident or survey exercise may be implemented as part of a regular monitoring process during any point of the food production line, or may be implemented to monitor for possible contamination events in response to a change in the food product supply chain, e.g., receiving raw materials from a new supplier. At 104, the incident or survey exercise prompts the implementation of the method at, for example, the factory level, at the supplier level, or for product testing purposes. The method can be implemented at one or more particular points during each level of the food production chain. Moreover, at each level in the food production chain, one or more food products may be evaluated. At 106, one or more samples are obtained from one or more locations with the food production environment at the designated testing level. The sample generation may also involve obtaining one or more origin samples from one or more potential origins of the one or more unwanted microbes, e.g., samples from one or more locations in the food product supply chain.

Once obtained, the one or more samples are processed at a designated location, such as an internal or external laboratory (108). Once the physical sample is received by the internal or external laboratory, the sample is processed at step 110. During sample processing, nucleic acids (e.g., DNA or RNA) are extracted from the physical sample, a sequencing library (e.g., a DNA or RNA library) is prepared, the library is analyzed (e.g., by loading the library onto a microarray or nucleic acid sequencer), and data is generated. Any known method for nucleic acid extraction and library preparation known in the art may be used. For instance, without limitation, nucleic acid extraction may be performed on freshly collected or frozen samples, and using any available extraction technique such as phenol:chloroform:isoamyl alcohol extraction or by using any appropriate commercially available kit. The sequencing library may be analyzed using any available technique that provides nucleic acid sequence data, such as, without limitation, next generation sequencing, qPCR, mass spectrometry, chromatography, microarray, in situ sequencing, probe hybridization, and any combination thereof. The method of sequencing library preparation will depend on the analysis technique to be used and may be performed according to the sequencing platform manufacturer's instructions. The generated data is transferred at 112 as incoming data (e.g., DNA or RNA sequence data) to a central location in the organization (114). The central location may be user accessible, such as a laptop, an external hard drive, a data lake or a cloud, or any other local or centralized system in the organization, or a data storage location available as a service to the organization. The transferred data may be provided from an internal laboratory or an external source, and may be stored in the central location until further downstream analysis.

At 116, the data from the central location is accessed by analytical platforms. The analytical platform may comprise one or more databases or software that enables analysis of the nucleic acid sequence data. Analysis of the nucleic acid sequences may comprise identifying one or more origin-indicating microbial signatures within the plurality of nucleic acid sequences and comparing the identified origin-indicating microbial signatures against one or more databases of origin-indicating microbial signatures that correspond to particular origins of unwanted microbes. Analysis of the nucleic acid sequences can further include, without limitation, comparing sequences against one or more additional databases; filtering sequence reads by size, quality, or origin; de-multiplexing a sample; sequence mapping; read quantification; or any combination thereof. Any suitable analytical platform, such as a platform comprising publicly available software or database, or in-house software or databases may be used. Analysis of the data using the analytical platforms results in one or more unwanted microbe origin determination outcomes (118). The one or more unwanted microbe origin determination outcomes may be included in internal or external reports, which may be reported as a physical report, or displayed in a user interface. For instance, a user interface may display the one or more unwanted microbe origin determination outcomes and allow a user to navigate and refine the outcomes. Additionally, the user interface may allow a user to compare one or more unwanted microbe origin determination outcomes corresponding to different samples from different locations in the food product supply chain, e.g., samples of materials from different suppliers, samples from different factories or transport vehicles, or samples from different farms. The user may likewise compare samples from clinical sources such as patients, equipment or other surfaces.

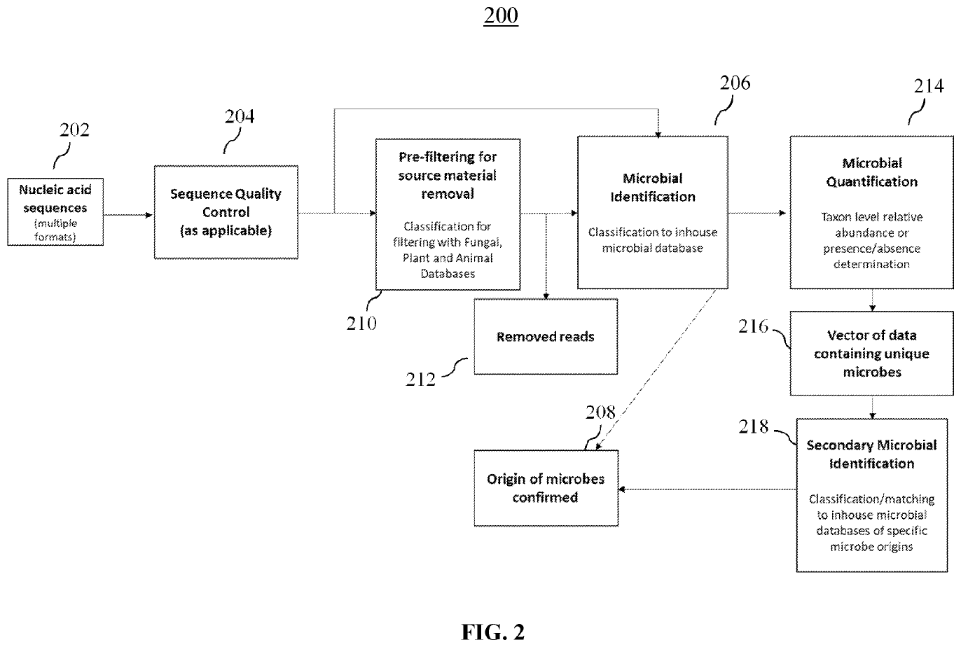

FIG. 2 depicts an exemplary analysis process for identifying one or more origin-indicating microbial signatures within a plurality of nucleic acid sequences from a sample, e.g., a food sample (method 200). Nucleic acid sequences are received at 202. The nucleic acid sequences may correspond to DNA, RNA, or both DNA and RNA sequences. The nucleic acid sequences may be provided in any suitable format. At 204, a sequence quality control may be implemented as applicable. The sequence quality control may include, without limitation, trimming, length filtering, sequencing adapter removal, sequence binning, or any combination thereof. The nucleic acid sequences are then analyzed for microbial identification (206), in which one or more origin-indicating microbial signatures are identified. Microbial identification may include classification to a microbial database (e.g., an in-house microbial database). The microbial databases may be specific to a particular category of microbes, such as a viral database or a fungal database, or may correspond to a wide range of microbes. The microbial databases may also correspond to microbes present in particular origins of unwanted microbes. Any suitable database corresponding to microbial sequences may be used for microbial identification. The databases may correspond to nucleic acid sequences consisting of combinations of nucleotides (e.g., A, T, G, or C), or they may correspond to amino acid sequences corresponding to one or more protein or protein isoforms encoded by microbial nucleic acids, and which may include any naturally and non-naturally occurring amino acid residues known in the art. After microbial identification, the origin of the unwanted microbe can be confirmed (208) based on the one or more origin-indicating microbial signatures. Prior to microbial identification, a pre-filtering step may be performed for removal of sequences corresponding to the non-microbial sequences, e.g., the source material of the sample matrix (210). The pre-filtering step can include classification of sequences using fungal, plant, or animal databases. In some instances, pre-filtering can identify sequence reads that do not correspond to any microbe present in the sample (e.g. unmapped sequences). At 212, the pre-filtered sequence reads may then be removed from the sequence data.

Microbial quantification (214) may also be performed. The microbial quantification may be determined as the taxon level relative abundance of one or more microbes, or as a presence or absence determination. Microbial quantification may be based on the number of reads corresponding to a particular microbe. For example, a higher read count for sequences corresponding to a particular microbe would indicate higher levels of that microbe in the food product. The quantification may be based on an internal or external control sample. In some instances, microbial quantification may include setting a threshold. For example, the presence or absence of a microbe may be determined based on whether the read count corresponding to that microbe is present in an amount meeting or exceeding a pre-determined threshold. Alternatively, the presence or absence of a microbe in a sample may be determined based on whether sequence reads from that sample surpass a pre-determined threshold, such as, at least 90%, at least 95%, or a 100% sequence identity match to at least one sequence in a reference database. Following microbial quantification, a vector data containing unique microbes is generated (216). The vector data is used for secondary microbial identification at 218. Secondary microbial identification may include, for example, classification or matching of the origin-indicating microbial signatures to one or more microbial databases. For example, in-house microbial databases corresponding to specific origins of unwanted microbes may be used for secondary microbial identification.

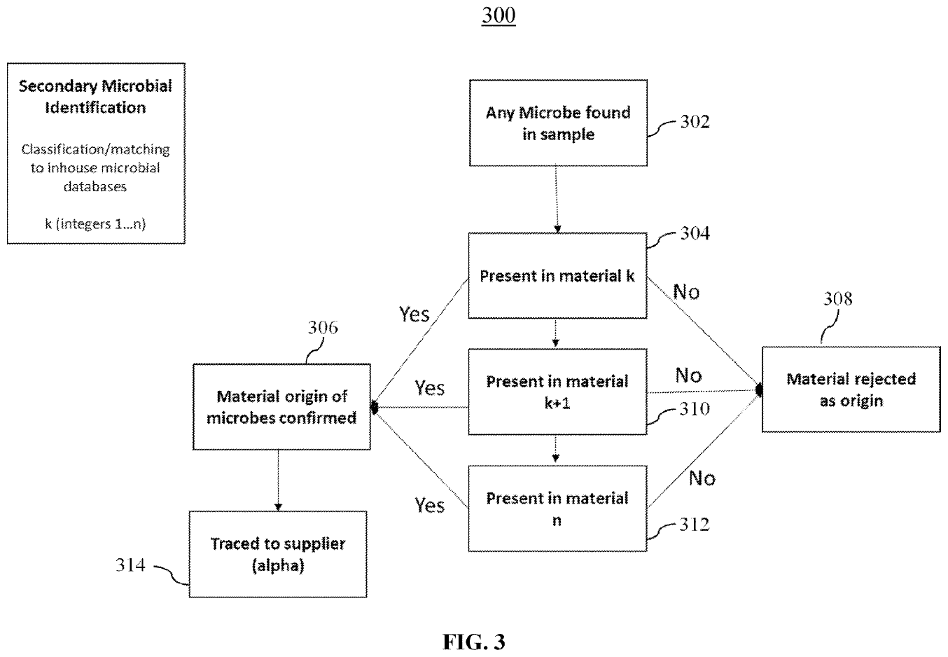

The methods described herein can comprise determining whether one or more origin-indicating microbial signatures in a food product sample correspond to one or more origins associated with a particular food source or a particular contaminant in a food production chain. FIG. 3 shows an exemplary process of determining whether origin-indicating microbial signatures correspond to one or more microbes associated with a particular origin of an unwanted microbe, e.g., particular food source materials (method 300). The method corresponds to secondary microbial classification and may involve classification or matching of origin-indicating microbial signatures to in-house microbial databases for specific origins of unwanted microbes k (integers k+1 . . . n). The determination is performed as an iterative process, where only one possible origin of the unwanted microbe is considered at each stage or step of the process. An origin-indicating microbial signature corresponding to any microbe found in the sample is received at 302. At 304, the microbial signature is analyzed to determine whether the microbe is present in origin material k. If the microbe is present in origin material k, the origin material is confirmed at 306. Alternatively, if the microbe is not present in origin material k, the origin material is rejected (308). The analysis is iterated to determine whether the microbe is present in origin material k+1. The origin material is confirmed (306) if the microbe is present in origin material k+1, but rejected (308) if the microbe is not present in origin material k+1. The analysis is further iterated for origin material n (312) with the origin material confirmed (306) if the microbe is present in origin material n, but rejected (308) if the microbe is not present in origin material n. Origins confirmed step 306 may be used for identifying the origin of an unwanted microbe in the sample, and may be traced to a particular point of entry into the food production chain, e.g., a particular supplier (314).

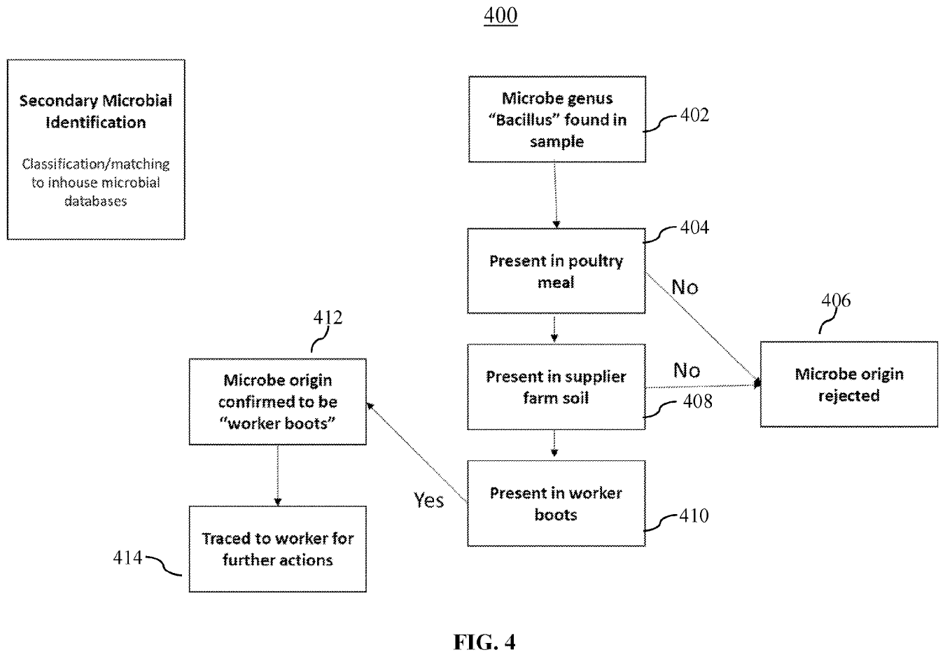

FIG. 4 depicts an example of secondary microbial identification for identifying the origin of an unwanted microbe in a food sample (method 400). Secondary microbial identification may be performed by classification or matching to in-house microbial databases of specific origins of unwanted microbes. The determination is performed as an iterative process, where only possible origin is considered at each stage or step of the process. In this example, an origin-indicating microbial signature associated with a microbe belonging to the genus Bacillus is found in the sample (402). At 404, the origin-indicating microbial signature is analyzed to determine whether the associated microbe is present in a poultry meal origin. Poultry meal is rejected as an origin at step 406 based on the determination that microbes belonging the genus Bacillus are not present in poultry meal. The origin-indicating microbial signature is then analyzed to determine whether the associated microbe is present in a second origin corresponding to a supplier farm soil (408). Supplier farm soil is rejected as an origin at 406 based on the determination that microbes belonging to the genus Bacillus are not present in the supplier farm soil. At 410, the origin-indicating microbial signature is then analyzed to determine whether the microbe is present in a third origin corresponding to food production worker boots. Based on the determination that microbes belonging to the genus Bacillus are present on the worker boots, the origin of the unwanted microbe is confirmed to be the worker boots (412). The confirmed origin can then be traced to the worker for corrective action.

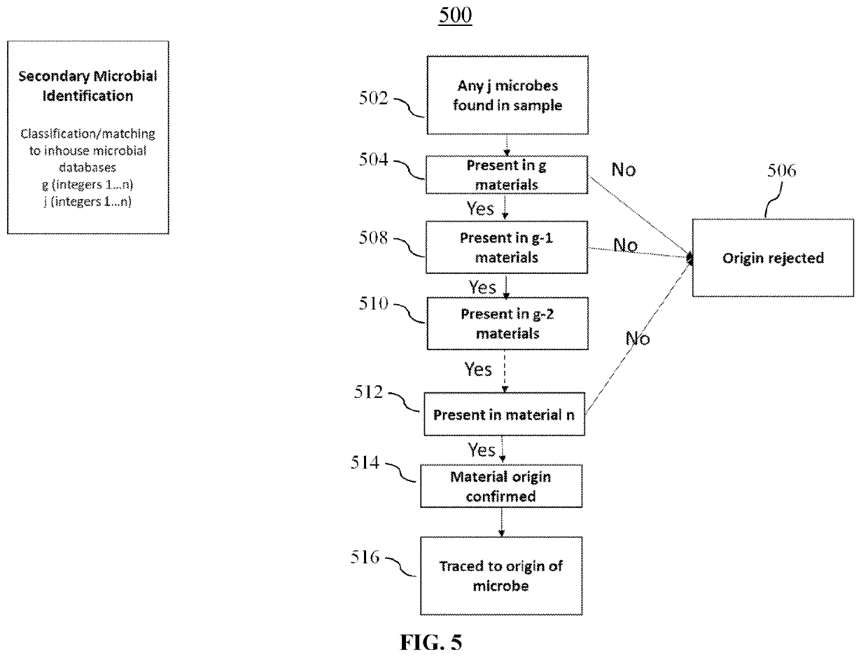

Multiple origin-indicating microbial signatures, each corresponding to one or more microbes present in the food product, can be analyzed to identify the origin of an unwanted microbe in an environment. FIG. 5 shows an exemplary method for secondary microbial identification for identifying the origin of an unwanted microbe using multiple microbial signatures corresponding to one or more microbes associated with particular origins (method 500). The method can involve classification or matching to in-house microbial databases of specific possible origins. In this method, multiple origin-indicating microbial signatures are considered in parallel, but only one particular material is considered as a possible origin at each stage. At each stage, a material is rejected as a possible origin if the origin-indicating microbial signatures do not correspond to one or more microbes associated with material being considered as a possible origin at that stage. The one or more microbes associated with each possible origin material g, g−1, g−2, etc., are different. The process may be repeated until all possible origin materials n have been considered. The determination may be performed in an iterative fashion, with a particular origin material considered at each different step of the process. Microbes corresponding to multiple origin materials may be considered first, or may be considered after microbes unique to a particular origin material have been first considered. Alternatively, the determination may be performed in parallel, with multiple origin materials being considered in parallel.

Microbial signatures corresponding to any j microbes present in the sample are received at step 502 and analyzed to obtain origin-indicating microbial signatures. At each of 504, 508, 510, and 512, the one or more origin-indicating microbial signatures obtained at 502 are analyzed to determine whether they correspond to any microbe j (integers 1 . . . n) associated with origin material g (integers 1 . . . n). For instance, at 504, the microbial signature is analyzed to determine whether any j microbes are present in g origin materials. The g origin materials are rejected (506) if none of the j microbes are present in the g origin materials. Alternatively, the g origin materials are confirmed if one or more of the j microbes are present in the g origin materials. After 504, the one or more origin-indicating microbial signatures are analyzed to determine whether the any j microbes are present in g−1 origin materials (508). The g−1 origin materials are rejected at 506, if none of the j microbes are present in the g−1 origin materials. Alternatively, the g−1 origin materials are confirmed if one or more of the j microbes are present in the g−1 origin materials. Similarly, at 510, the microbial signature is analyzed to determine whether any j microbes are present in g−2 origin materials. The g−2 origin materials are confirmed if one or more of the j microbes are present in the g−2 origin materials. The process is iterated for n origin materials at 512, with the origin-indicating microbial signature analyzed to determine whether any j microbes are present in n origin materials. The n origin materials are rejected at step 506, if none of the j microbes are present in the n origin materials. If one or more of the j microbes are present in the n origin materials, the n origin materials are confirmed. At 516, any confirmed origin material(s) are traced to a supplier.

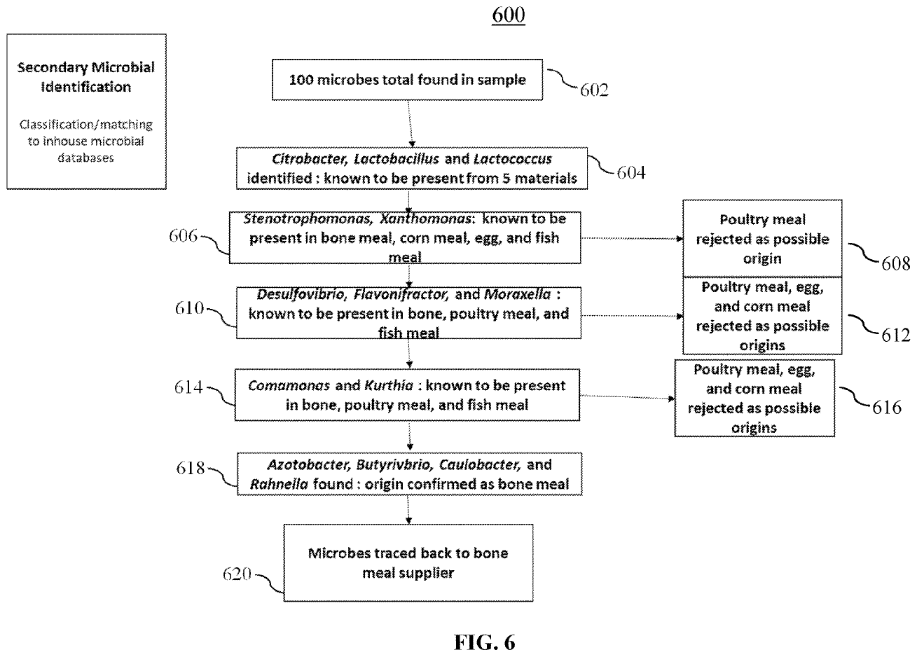

FIG. 6 shows an example of secondary microbial identification for identifying the origin of an unwanted microbe in an environment using multiple origin-indicating microbial signatures identified in a sample (method 600). The secondary microbial classification can be performed by classification or matching of origin-indicating microbial signatures to in-house microbial databases of specific possible origin materials. Microbial signatures corresponding to 90 total microbes found in the food sample are received at 602. At 604, microbes belonging to the Citrobacter, Lactobacillus, and Lactococcus genera are identified. These microbes are known to present in all five possible origin materials analyzed in this exemplary embodiment (bone meal, corn meal, egg, fish meal, and poultry meal). At 606, microbes from the Stenotrophomonas and Xanthomonas genera are identified. These microbes are known to be present in bone meal, corn meal, egg, and fish meal, but known to not be present in chicken meal. Chicken meal is rejected as a possible origin at 608. At 610, microbes belonging to the Desulfovibrio, Flavonifractor, and Moraxella genera are identified. Microbes from there genus are known to be present in bone meal, poultry meal, and fish meal. Poultry meal, egg, and corn meal are rejected as possible origins at step 612. At step 614, microbes belonging to the Comamonas and Kurthia genera are identified. These microbes are known to be present in bone meal, poultry meal, and fish meal. Poultry meal, egg, and corn meal are rejected as possible origins at 616. Microbes belonging to the Azotobacter, Butyrivbio, Caulobacter, and Rahnella genera are identified. Microbes from these genera are known to be present in bone meal. Accordingly, at step 618, the origin of the unwanted microbe is confirmed to be bone meal. The contamination event that introduced the unwanted microbe is then traced to a bone meal supplier at 620.

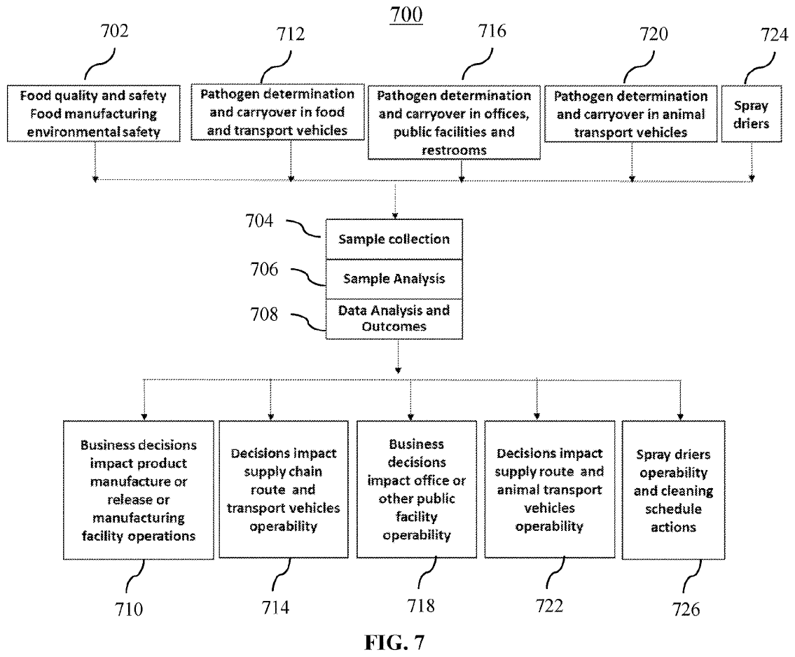

FIG. 7 shows examples of how the methods described herein can be applied to identify unwanted microbes, and/or one or more origins thereof, in a variety of environments and lead to corrective actions in such environments (flowchart 700). At 702, the method is initiated in a food manufacturing environment to promote food quality and safety, as well as safety of the food manufacturing environment. Samples are collected from one or more locations in the food manufacturing environment (704), the samples are analyzed for indicator microbial signatures and origin-indicating microbial signatures (706), and analysis of the microbial signatures leads to identification of unwanted microbes, and/or one or more origins thereof, in the environment (708). As a result, at 710, business decisions are made to alter product manufacture and release and/or manufacturing facility operations to correct identified contamination events and prevent future occurrence of contamination events. In an alternate example, a clinical decision to introduce corrective actions or prophylaxis may be arrived at.

At 712, the method is initiated to identify pathogens and trace-back to one or more origins thereof in a food production supply chain, including in food transport vehicles. Samples are collected from one or more locations in the food production supply chain, including in any food transport vehicles (704), the samples are analyzed for indicator microbial signatures and origin-indicating microbial signatures (706), and analysis of the microbial signatures leads to identification of unwanted microbes, and/or one or more origins thereof, in the food production supply chain (708). As a result, at 714, decisions are made that impact the supply chain route and operability of food transport vehicles to correct identified contamination events and prevent the future occurrence of such events.

At 716, the method is initiated to identify pathogens and trace-back to one or more origins thereof in offices, public facilities, and restrooms. Samples are collected from one or more locations in the office, public facilities, and/or restrooms (704), the samples are analyzed for indicator microbial signatures and origin-indicating microbial signatures (706), and analysis of the microbial signatures leads to identification of unwanted microbes, and/or one or more origins thereof, in the office, public facilities, and/or restrooms (708). As a result, at 718, business decisions are made that impact the management of the office, public facility, and/or restroom, to correct identified contamination events and prevent the future occurrence of such events.

At 720, the method is initiated to identify pathogens and trace-back to one or more origins thereof in a livestock production environment, including in animal transport vehicles. Samples are collected from one or more locations in the livestock production environment, including in any animal transport vehicles (704), the samples are analyzed for indicator microbial signatures and origin-indicating microbial signatures (706), and analysis of the microbial signatures leads to identification of unwanted microbes, and/or one or more origins thereof, in the livestock production environment (708). As a result, at 722, decisions are made that impact the livestock production supply chain route and operability of livestock transport vehicles to correct identified contamination events and prevent the future occurrence of such events.

At 724, the method is initiated to identify pathogens and trace-back to one or more origins thereof in a spray drier. Samples are collected from one or more locations in and/or around the spray dryer (704), including the interior and exterior of the spray dryer and any materials fed into or produced by the spray drier. The samples are then analyzed for indicator microbial signatures and origin-indicating microbial signatures (706), and analysis of the microbial signatures leads to identification of unwanted microbes, and/or one or more origins thereof, in the spray drier or the surrounding environment (708). As a result, in 726, actions are taken in spray drier operations to correct identified contamination events and prevent the future occurrence of such events.

The methods described herein may be used to detect unwanted microbes, and/or identify one or more origins thereof, in a wide range of environments, including, but not limited to, food production environments (e.g., a farm, a food transport vehicle, an animal transport vehicle, a food processing facility, a food packaging facility, a food distribution facility, a warehouse, restaurant and a food market), environments in public buildings (e.g., a public restroom), environments in medical buildings (e.g. clinics or hospitals), environments in office buildings (e.g., an office), environments in residential buildings, or environments in pet or animal care facilities (e.g., a veterinary office, a pet hospital, a kennel, a zoo enclosure or a pet daycare facility). Using the methods described above, one or more different origins of unwanted microbes (e.g., contamination events) may be identified for a particular environment (e.g., a food product, a food production facility, a public restroom, or a spray drier). In some embodiments, the methods may also be performed at multiple steps within a production chain, e.g., a food product production chain or a livestock production chain. The methods described herein may comprising collecting samples from two three, four, five, ten, or more locations within an environment. Additionally, the methods described herein may comprise collecting samples from two, three, four, five, ten, or more environments. To detect and/or identify the origin of unwanted microbes in a food production environment, the method may comprise obtaining one or more samples from various locations in a food production chain including, but not limited to, agricultural or farm soil, agricultural or farm equipment, crops, livestock, transport vehicle surfaces, factory equipment, factory floors, food processing material, food processing equipment, food products, worker apparel, or any combination thereof. To detect and/or identify the origin of unwanted microbes in a public building, office building, a residential building, or animal care facility environment, the method may comprise obtaining one or more samples from various locations in the public building, office building, a residential building, or animal care facility environment including, for example, a companion animal (e.g., a dog or a cat), a service animal (e.g., a service dog or a service horse), a zoo or wild animal (e.g., a fish, coral, whale, tiger or elephant) or an item or surface contacted by a companion animal or a service animal (e.g., a floor, a veterinary table, a collar, a leash, a harness, a furniture item, or a rug).

The methods described herein may be used to detect one or more unwanted microbes in spray driers and determine the origin(s) of the unwanted microbes. The method may comprise obtaining one or more samples from one or more locations within the spray dryer, from a powder produced by the spray dryer (e.g., milk powder), or from a liquid to be fed into the spray dryer (e.g., milk), or any combination thereof associated with the one or more unwanted microbes in the spray dryer, around the spray dryer, or both. In some instances, two, three, four, five, ten, or more samples are collected from two, three, four, five, ten, or more locations within the spray dryer or on the surfaces of the spray dryer. Samples may be taken from any location within the spray dryer, on the surface of the spray dryer, or in the vicinity of the spray drier, including, but not limited to, the inlet, the outlet, the storage silos, the drying chamber, or the cyclone of the spray dryer, or any combination thereof. In some cases, where the spray dryer is used for producing milk powder, samples are obtained from the liquid milk to be fed into the spray drier or the milk powder produced by the spray drier.

Similar methods as those disclosed herein may be used to detect the presence of a contaminating unwanted microbe in an environment. The indicator microbial signatures and/or origin-indicating microbial signatures may correspond to microbes associated with a particular environmental contaminant. The microbial signatures can then be analyzed by comparing to a database of microbes associated with particular environmental contaminants, thus allowing the detection of the environmental contaminant, and trace-back of the contaminant to its point of origin in the environment.

In a food production environment, a particular contaminant may also be traced back to a particular supplier, e.g., a supplier of raw materials used in a food production process. For example, contamination of the food product during food production may be detected and traced back to a particular supplier. For example, after analysis of nucleic acid sequences from a chicken food product, an indicator microbial signature may be identified that indicates an unwanted microbe present in the sample, and an origin-indicating microbial signature may be identified that indicates pork as the origin of the unwanted microbe. The origin-indicating microbial signature may then be traced back to a specific point in the food production chain. The pork origin may correspond, for instance, to a contaminating pork food material or to a particular location in the supply chain that processes pork. The pork contaminant can then be matched to a food material provided by a particular supplier, or to a location of a specific supplier for a particular step of the food production chain. The supplier for the food material is then identified, and after some investigation, the supplier may find, for example, that the chicken raw materials were stored in the same transporting unit as pork raw materials. The presence of the pork contamination would lead to deviations in the microbiome of the food product that could be detected, even after the chicken material was no longer in close proximity to the food material. The food producer may then consider that particular source of chicken as compromised. The producer may then decide to implement a corrective action, such as issuing a warning to the supplier or changing to a different supplier altogether. In this way, the methods disclosed herein may be used for end-to-end trace-back of contaminants during the food production process. By analyzing samples for indicator microbial signatures and origin-indicating microbial signatures at one or more steps of the food production chain, the producer can make sure that there is no contamination of unwanted microbes introduced into the food product.

### II. Nucleic Acid Sequencing

The methods disclosed herein comprise sequencing a plurality of nucleic acid sequences within the one or more samples. The nucleic acid sequences may correspond to any nucleic acid present in a sample. For instance, the nucleic acid sequences may correspond to a plurality of DNA and/or RNA sequences. The plurality of nucleic acid sequences may correspond to nucleic acids from one or more microbes present in sample, or to nucleic acids from the sample matrix, e.g., the food matrix material in a food product sample.

Sequencing a plurality of nucleic acid sequences within the one or more samples can include extracting nucleic acids from the sample. Methods of extracting nucleic acids known in the art may be used. Without being limited, nucleic acids may be extracted using TrizolLS reagent, phenol:chloroform:isoamyl alcohol extraction, or equivalents. Nucleic acid extraction may also be performed using commercially available kits, such as, Ambion RNA isolation kits (e.g., Purelink RNA Mini kit or DynaBeads mRNA direct micro kit), MAgmax FFPE total nucleic acid isolation kit, Pall DNA and RNA Purification kits, Qiagen Allprep, PowerViral, Powersoil, or PowerMag kits, NEBNext Microbiome DNA Enrichment kit, or equivalents. Nucleic acid extraction may be performed using frozen or fresh samples. For example, a food product may be fixed before nucleic acid extraction. Nucleic acid extraction may also include a step of cell lysis. Cell lysis may be performed through any methods known to those skilled in the art, including, but not limited to, enzymatic lysis using lytic enzymes such as lysozyme, lysostaphin, mutanolysin, proteinase K, subtilisin, or any combination thereof; physical shearing, such as with glass beads, sonication, ultrasound, or high pressure (e.g., using French press); and any other cell lysis method known to those skilled in the art.

It should be understood that the present teachings contemplate sequencing a plurality of nucleic acid sequences within the one or more samples using all available varieties of techniques, platforms, or technologies, including, but not limited to: capillary electrophoresis, microarrays, ligation-based systems, polymerase-based systems, hybridization-based systems, in situ sequencing, direct or indirect nucleotide identification systems, pyrosequencing, ion- or pH-based detection systems, electronic signature-based systems, etc.

The plurality of nucleic acid sequences within the one or more samples may be sequenced by any method available in the art, such as by nucleic acid sequencing (e.g., next generation sequencing) or microarray analysis. The methods disclosed herein are not dependent upon a particular next generation sequencing technology, and the user needs to make appropriate choices for the intended downstream sequencing platform according to manufacturers' protocols. Exemplary sequencing platforms that may be used to obtain sequence data according to the methods disclosed herein include, but are not limited to, those produced by Illumina®, Oxford Nanopore™, Jon Torrent™, Roche™, Pacific Biosciences™ and Life Technologies™.

Depending on the sequencing technology used with the methods, a sequencing library may be prepared. The sequencing library will be representative of nucleic acids present in a sample and can be used with next generation sequencing platforms. Sequencing library preparation can include nucleic acid fragmentation, sample indexing, adaptor ligation, and library normalization. Sample indexing or barcoding allows multiple samples to be run simultaneously, taking full advantage of the high-throughput nature of current sequencing platforms. Adapter ligation is sequencing platform specific and standard to manufacturers' protocols. The adaptors may contain sequencing platform-specific end sequences and index sequences that allow for de-convolution of sequence data by sample. Barcoding and adapter ligation may be performed by any method known to those in the art, and may be adapted for analysis of the sequencing library with a particular sequencing platform. Library preparation can also include amplification, concentration, or dilution of the sequencing library. Libraries can be prepared at platform-specific concentrations of DNA or RNA and typically require amplification, concentration, or dilution to achieve the required concentration. The concentration of nucleic acids in the sequencing library may be determined by quantitative real-time PCR using platform specific manufacturer protocols or fluorescence-based measurement known in the art. In some instances, preparing the sequencing library includes selective enrichment of specific target nucleic acids or regions.

Nucleotide sequences of individual molecules are determined in a platform-specific manner to produce a raw dataset. The raw dataset can be converted to nucleotide sequencing information corresponding to each molecule in a sequencing library. The resulting products are whole “reads,” which may be processed to determine information about the food product. The sequence data may be produced in any format, such as BAM files, which are sequencing platform-independent and ready for bioinformatics analysis. Additional file types may include FASTA and FASTQ file formats, or other manufacturer-specific formats that can be converted to BAM, VCF, FASTQ, or FASTA format.

Once obtained, sequence data containing the plurality of nucleic acid sequences may be transferred in real time from the instrument used to generate sequence data as soon as the sequence data has reached a sufficient size in total base pairs for analysis, or it may be stored in a database until further analysis. Data may be stored on any suitable database or device, including, but not limited to, on a server, on a personal computer, on a smartphone, on a tablet, on a cloud system (e.g., AWS™, Google Cloud™, or MS Azure™), or on a local hard drive (e.g., an external hard drive).

The sequence data containing the plurality of nucleic acid sequences may then be prepared for further analysis. This preparation can include performing sequence quality control, trimming, length filtering, sequencing adapter removal, and/or binning of reads by molecular barcode from the sequencing reads. In particular, the reads that represent the plurality of nucleic acid sequences from the sample can be quality controlled to remove the adapter sequences, clonal reads due to PCR amplification, and platform-specific sequence errors and filtered to achieve an acceptable error rate. Sequencing reads in the sequencing data may be deconstructed into, for example, k-mers of a particular size. Sequence assembly, mapping, or pairwise comparison of the sequencing reads in the sequence data may also be performed. In some cases, nucleic acid sequences corresponding to the sample matrix or other agent can be filtered or removed from the sequence data prior to further analysis. For example, if the sample is a food product sample, nucleic acid sequences corresponding to the food material matrix can be removed from the sequence data prior to further analysis.

### III. Indicator Microbial Signatures

The methods described herein can include identifying one or more indicator microbial signatures within the plurality of nucleic acid sequences in the one or more samples. The one or more indicator microbial signatures may correspond to one or more microbes present in the sample and may indicate the presence of one or more unwanted microbes present in the sample. Importantly, the indicator microbial signature may correspond to one or more microbes present in the sample that do not include the unwanted microbe. For example, an unwanted microbe (e.g., a pathogenic bacterium) may be present in the sample in an amount insufficient to be detected by direct sequencing of the unwanted microbe. In such a case, the indicator microbial signature may correspond to one or more microbes other than the unwanted microbe, e.g., a set of bacterial species present in a relative abundance that indicates a perturbation in the normal microbial community caused by the introduction of the unwanted microbe. Therefore, as used herein, an indicator microbial signature “corresponds to” a given microbe present in the sample when the indicator microbial signature includes sequences of the given microbe. By contrast, an indicator microbial signature can “indicate” the presence of an unwanted microbe even if it does not include sequences of the unwanted microbe. The indicator microbial signatures may correspond to a particular taxonomic level, such as a kingdom, a phylum, a class, a genus, a species, a serotype, or a strain. Additionally, the indicator microbial signature may indicate an unwanted microbe of a particular taxonomic level, such as a kingdom, a phylum, a class, a genus, a species, a serotype, or a strain. The microbes present in the sample (including any unwanted microbes) may be microbial eukaryotes, bacteria, archaea, fungi, or viruses.

Identifying the one or more indicator microbial signatures can include comparing sequence data to one or more databases. The databases can contain sequences (e.g., nucleic acid sequences, or amino acid sequences) from a particular group of microbes. For instance, the databases may correspond to nucleic acid sequences from microbes that may indicate the presence of a particular unwanted microbe. These databases may correspond to a specific microbial taxon, a specific genus, or a collection of microbial species. Any publicly available database that is suitable for microbe identification may be used. Alternatively, an in-house database may be generated and used to identify a microbial signature.

Identifying one or more indicator microbial signatures can include determining the relative level of nucleic acids in the sequence data corresponding to one or more particular microbes in the sample. The relative level of the nucleic acids in the indicator microbial signature can be indicative of the relative level of the particular microbes and may be used to determine the relative abundance of particular microbes. A threshold may be set, and any indicator microbial signature corresponding to a relative level of one or more microbes above the predetermined threshold would indicate the presence and/or abundance of an unwanted microbe. The threshold may be set in terms of, for example, a Ct value, a nucleic acid copy number, a concentration (e.g., in mg/mL or mg/L units), etc.

Indicator microbial signatures may correspond to one or more of a bacteria, virus, archaea, or eukaryotic microorganisms. Exemplary microbes that can be present in a sample include, but are not limited to, microbes belonging to a genus taxonomy selected from the group consisting of Parageobacillus, Blautia, Ahiivibrio, Porphyrobacter, Shigella, Aneurinibacillus, Anaerostipes, Photobacterium, Erythrobacter, Rathayibacter, Butyrivibrio, Tyzzerella, Grimontia, Dechloromonas, Leifsonia, Coprothermobacter, Intestinimonas, Pseudoalteromonas, Pseudarthrobacter, Arthrobacter, Megasphaera, Ethanoligenens, Alteromonas, Isoptericola, Micrococcus, Eubacterium, Colwellia, Cellulomonas, Thermus, Oscillibacter, Yersinia, Nocardia, Meiothermus, Weissella, Edwardsiella, Gordonia, Rahnella, Murdochiella, Oceanimonas, Propionibacterium, Azotobacter, Eggerthella, Marinomonas, Tessaracoccus, Caulobacter, Adlercreutzia, Halomonas, Pimelobacter, Fibrobacter, Gordonibacter, Methylophaga, Actinoplanes, Fervidobacterium, Obesumbacterium, Brucella, Listeria, Methanobrevibacter, Plesiomonas, Caldanaerobacter, Deinococcus, Methanosarcina, Gallibacterium, Synechococcus, Spirosoma, Thioploca, Calothrix, Helicobacter, Thermotoga, Janthinobacterium, Nonlabens, Barnesiella, Fusobacterium, Ornithobacterium, Ilyobacter, Akkermansia, Thermodesulfobacterium, Cloacibacillus, Theileria, Gyrovirus, T7virus, T4virus, Alpharetrovirus, Sp18virus, Acidaminococcus, Altererythrobacter, Comamonas, Arcobacter, Aeromicrobium, Pediococcus, Proteus, Alistipes, Azospira, Geobacillus, Geoalkalibacter, Agrobacterium, Vibrio, Christensenella, Bosea, Kurthia, Hafnia, Alcaligenes, Clostridioides, Novosphingobium, Oblitimonas, Morganella, Amycolatopsis, Odoribacter, Pseudoxanthomonas, Negativicoccus, Aureimonas, Olsenella, Psychrobacter, Paenibacillus, Brachybacterium, Parabacteroides, Shewanella, Providencia, Brevibacterium, Roseburia, Candida, Ruminococcus, Caulimovirus, Selenomonas, Clavibacter, Treponema, Curtobacterium, Turicibacter, Erwinia, Frondihabitans, Hymenobacter, Kineococcus, Kluyveromyces, Massilia, Methylobacterium, Microbacterium, Nocardioides, Ochrobactrum, Pseudonocardia, Rhizobium, Saccharopolyspora, Sanguibacter, Shinella, Sphingobacterium, Sugiyamaella, Chryseobacterium, Aeromonas, Achromobacter, Blastomonas, Pantoea, Delftia, Anoxybacillus, Bordetella, Mycobacterium, Bacteroides, Brevundimonas, Rhodococcus, Bifidobacterium, Kosakonia, Streptomyces, Desulfovibrio, Sphingobium, Thermothelomyces, Flavonfractor, Sphingomonas, Thielavia, Lachnoclostridium, Sphingopyxis, Macrococcus, Cupriavidus, Moraxella, Prevotella, Ruminiclostridium, Bradyrhizobium, Campylobacter, Clostridium, Stenotrophomonas, Burkholderia, Cutibacterium, Xanthomonas, Serratia, Escherichia, Staphylococcus, Streptococcus, Variovorax, Acidovorax, Acinetobacter, Bacillus, Citrobacter, Corynebacterium, Enterobacter, Enterococcus, Klebsiella, Lactobacillus, Lactococcus, Pseudomonas, Raoultella, and Salmonella. An indicator microbial signature may correspond to any microbe of the foregoing taxonomic designations, either singly or in any combination. Additionally, any indicator microbial signature may indicate the presence of an unwanted microbe of any of the foregoing taxonomic designations.

### IV. Origin-Indicating Microbial Signatures

The methods described herein can include identifying one or more origin-indicating microbial signatures within the plurality of nucleic acid sequences. The one or more origin-indicating microbial signatures may correspond to one or more microbes present in the sample and may indicate the origin of one or more unwanted microbes present in the sample. Importantly, the origin-indicating microbial signature may correspond to one or more microbes present in the sample that do not include the unwanted microbe. For example, an unwanted microbe (e.g., a pathogenic bacterium) may be present in the sample, but the presence of the unwanted microbe alone may be insufficient to identify the origin of the unwanted microbe. In such a case, the origin-indicating microbial signature may correspond to one or more microbes other than the unwanted microbe, e.g., a set of bacterial species present in a relative abundance that indicates a perturbation in the normal microbial community caused by the introduction of a contaminant containing the unwanted microbe. Therefore, as used herein, an origin-indicating microbial signature “corresponds to” a given microbe present in the sample when the origin-indicating microbial signature includes sequences of the given microbe. By contrast, an indicator microbial signature can “indicate” the origin of an unwanted microbe even if it does not include sequences of the unwanted microbe. The origin-indicating microbial signatures may correspond to may correspond to one or more microbes a particular taxonomic level, such as a kingdom, a phylum, a class, a genus, a species, a serotype or a strain. The microbes present in the sample (including any unwanted microbes) may be microbial eukaryotes, bacteria, archaea, fungi, or viruses.

Identifying the one or more origin-indicating microbial signatures can include comparing sequence data to one or more databases. The databases can contain sequences (e.g., nucleic acid sequences, or amino acid sequences) from a particular group of microbes. For instance, the databases may correspond to nucleic acid sequences from microbes associated with particular origins of unwanted microbes, e.g., potential contaminating materials or equipment in an environment. These databases may correspond to a specific microbial taxa, a specific genus, or a collection of microbial species associated with particular origins of unwanted microbes. Any publicly available database that is suitable for microbe identification may be used.

In some cases, an in-house database may be generated and used to identify an origin-indicating microbial signature. Generating the database to identify origin-indicating microbial signatures may comprise obtaining samples of possible origins and detecting the unwanted microbe in the origin samples. For example, in some embodiments, identifying the one or more origin-indicating microbial signatures can comprise obtaining one or more (e.g., one, two, three, four, five, or ten) origin samples from one or more (e.g., one, two, three, four, five, or ten) potential origins of the one or more unwanted microbes; sequencing a plurality of origin nucleic acid sequences within the one or more origin samples; detecting one or more nucleic acid sequences of the one or more unwanted microbes in one or more of the origin samples; and identifying one or more origins of the one or more unwanted microbes based on detecting the one or more nucleic acid sequences of the one or more unwanted microbes in one or more of the origin samples. In some instances, the method further comprises identifying an unknown origin-indicating microbial signature within the plurality of origin nucleic acids sequences of the one or more origin samples in which the one or more unwanted microbes were detected, and optionally adding the unknown origin-indicating microbial signature and the corresponding origin of unwanted microbes to the one or more databases.

Identifying one or more origin-indicating microbial signatures can include determining the relative level of nucleic acids in the sequence data corresponding to one or more particular microbes in the sample. The relative level of the nucleic acids in the origin-indicating microbial signature can be indicative of the relative level of the microbes and may be used to determine the likelihood or extent of a contamination event from a given origin. A threshold may be set, and any microbial signature corresponding to a relative level of microbes above the predetermined threshold would be indicative of a contamination event in which the indicated origin resulted in the introduction of the unwanted microbe into the environment. The threshold may be set in terms of, for example, a Ct value, a nucleic acid copy number, a concentration (e.g., in mg/mL or mg/L units), etc.

In a food production environment, the microbes present in the sample may be associated with a particular food material origin or contaminant. The origin of the unwanted microbe may be of fungal, plant, or animal origin. Plant origins include, but are not limited to, grains (e.g., corn, rice, wheat), legumes (e.g., soy, peanut, chickpeas, lima beans, and kidney beans), tree nuts (e.g., cashews, walnuts, pecans, and hazel nuts), or any variety thereof, or any other plant origins, such as domesticated or wild plant crops, that may be used in food production. Corn meal is a commonly used food material of plant origin and may be an origin of an unwanted microbe. Fungal origins include, but are not limited to, yeasts, molds, and mushrooms. Without limitation, animal origins may be from fish, beef, goat, egg, pork, poultry, dairy, or shellfish. Animal origins of unwanted microbes include various meats, such as canine, feline, equine, bovine, or ovine meats, or fowl-borne meats such as but not limited to turkey, chicken, duck, or goose, or meat from any other animal, such as from a game animal. Animal origins may also include egg, poultry meal, fish meal, and bone meal, and other commonly used raw materials of animal origin. Animal origins of unwanted microbes may also include animal origins other than meat and animal meals, for example, animal feces, urine, hair, blood, or other fluids that may be accidentally introduced during the food production chain. Any other animal food source suitable for food production may also be identified as an origin of an unwanted microbe.

In a food production environment, the microbes present in the sample may be associated with a particular object, location, equipment, or other material in the food production environment. For example, the origin of the unwanted microbe may be an agricultural soil, an agricultural equipment surface, a crop, a livestock animal, a transport vehicle surface, a factory equipment surface, a factory floor, a food processing material, a food processing equipment surface, a food product, a worker apparel, or any combination thereof. In some embodiments, the one or more origins of the one or more unwanted microbes comprise an origin that corresponds to a particular geographical region.

In a public building, office building, residential building, clinic, or animal care facility environment, the microbes present in the sample may be associated with a particular wild animal, domestic animal, such as a livestock animal, a companion animal, or a service animal. Domestic animals may include, for example, domestic dogs, cats, horses, cows, ferrets, rabbits, pigs, rats, mice, gerbils, hamsters, horses, birds (e.g., parrots and parakeets), and the like. Animal origins of unwanted microbes can include any wild or domestic animal known in the art or described herein. In some embodiments, the animal origin is a companion animal or pet. In other embodiments, the animal origin is a service animal (e.g., a service dog or a service horse). In other embodiments, the animal origin is a wild or zoo animal. Animal origins of unwanted microbes may also include wild and domestic animal feces, urine, hair, blood, or other fluids that may be accidentally introduced into the environment.

In some instances, the one or more origin-indicating microbial signatures correspond to one or more microbes associated with a particular geographical region. Without being limited by theory, a particular microbe may be present in a particular region due to the presence of certain favorable conditions (e.g., soil composition, climate, temperature, altitude, topography, human management, etc.). However, the same microbe may be absent or rarely observed outside of that geographical region, and thus will be detected mainly in food materials from that geographical region. For example, a particular microbe or a particular species of this microbe may be detected in a plant cultivar from one particular region but will not be detected from a similar cultivar in a different region. The presence of the microbe or particular species in a food product would indicate that the raw materials were sourced from the particular region were the microbe can be found, and thus that the geographical location could be an origin of an unwanted microbe in the sample. The geographical region may be limited to a particular farm or field, or may extend to a bigger geographical are sharing the same topology and climate.

Origin-indicating microbial signatures may correspond to one or more of a bacteria, virus, archaea, or eukaryotic microorganisms. Exemplary microbes that can be present in a sample include, but are not limited to, microbes belonging to a genus taxonomy selected from the group consisting of Parageobacillus, Blautia, Aliivibrio, Porphyrobacter, Shigella, Aneurinibacillus, Anaerostipes, Photobacterium, Erythrobacter, Rathayibacter, Butyrivibrio, Tyzzerella, Grimontia, Dechloromonas, Leifsonia, Coprothermobacter, Intestinimonas, Pseudoalteromonas, Pseudarthrobacter, Arthrobacter, Megasphaera, Ethanoligenens, Alteromonas, Isoptericola, Micrococcus, Eubacterium, Colwellia, Cellulomonas, Thermus, Oscillibacter, Yersinia, Nocardia, Meiothermus, Weissella, Edwardsiella, Gordonia, Rahnella, Murdochiella, Oceanimonas, Propionibacterium, Azotobacter, Eggerthella, Marinomonas, Tessaracoccus, Caulobacter, Adlercreutzia, Halomonas, Pimelobacter, Fibrobacter, Gordonibacter, Methylophaga, Actinoplanes, Fervidobacterium, Obesumbacterium, Brucella, Listeria, Methanobrevibacter, Plesiomonas, Caldanaerobacter, Deinococcus, Methanosarcina, Gallibacterium, Synechococcus, Spirosoma, Thioploca, Calothrix, Helicobacter, Thermotoga, Janthinobacterium, Nonlabens, Barnesiella, Fusobacterium, Ornithobacterium, Ilyobacter, Akkermansia, Thermodesulfobacterium, Cloacibacillus, Theileria, Gyrovirus, T7virus, T4virus, Alpharetrovirus, Sp8virus, Acidaminococcus, Altererythrobacter, Comamonas, Arcobacter, Aeromicrobium, Pediococcus, Proteus, Alistipes, Azospira, Geobacillus, Geoalkalibacter, Agrobacterium, Vibrio, Christensenella, Bosea, Kurthia, Hafnia, Alcaligenes, Clostridioides, Novosphingobium, Oblitimonas, Morganella, Amycolatopsis, Odoribacter, Pseudoxanthomonas, Negativicoccus, Aureimonas, Olsenella, Psychrobacter, Paenibacillus, Brachybacterium, Parabacteroides, Shewanella, Providencia, Brevibacterium, Roseburia, Candida, Ruminococcus, Caulimovirus, Selenomonas, Clavibacter, Treponema, Curtobacterium, Turicibacter, Erwinia, Frondihabitans, Hymenobacter, Kineococcus, Kluyveromyces, Massilia, Methylobacterium, Microbacterium, Nocardioides, Ochrobactrum, Pseudonocardia, Rhizobium, Saccharopolyspora, Sanguibacter, Shinella, Sphingobacterium, Sugiyamaella, Chryseobacterium, Aeromonas, Achromobacter, Blastomonas, Pantoea, Delftia, Anoxybacillus, Bordetella, Mycobacterium, Bacteroides, Brevundimonas, Rhodococcus, Bifidobacterium, Kosakonia, Streptomyces, Desulfovibrio, Sphingobium, Thermothelomyces, Flavonfractor, Sphingomonas, Thielavia, Lachnoclostridium, Sphingopyxis, Macrococcus, Cupriavidus, Moraxella, Prevotella, Ruminiclostridium, Bradyrhizobium, Campylobacter, Clostridium, Stenotrophomonas, Burkholderia, Cutibacterium, Xanthomonas, Serratia, Escherichia, Staphylococcus, Streptococcus, Variovorax, Acidovorax, Acinetobacter, Bacillus, Citrobacter, Corynebacterium, Enterobacter, Enterococcus, Klebsiella, Lactobacillus, Lactococcus, Pseudomonas, Raoultella, and Salmonella. An origin-indicating microbial signature may correspond to any microbe of the foregoing taxonomic designations, either singly or in any combination.

In a food production environment, the origin-indicating microbial signatures can correspond to one or more microbes associated with one origin, e.g., one food source or contaminant. Microbes associated with one food source or contaminant include, but are not limited to, the microbes listed in Table 1 of the examples. In some instances, the origin-indicating microbial signatures can correspond to one or more microbes associated with two or more origins, e.g., two or more food sources or contaminants. Without limitation, microbes associated with two or more food sources or contaminants include those listed in Tables 2-5 of the examples. The microbes may be associated with at least two origins, or they may be associated with more than two origins, such as three or four origins.

### V. Systems

In one aspect, the disclosure provides systems for performing any of the methods described herein.

In some embodiments, the system can be configured to detect an unwanted microbe in an environment. For example, the system may include one or more processors and a memory comprising instructions executable by the one or more processors. When executed by the one or more processors, the instructions may cause the system to identify one or more indicator microbial signatures within a plurality of nucleic acid sequences obtained from sequencing a plurality of nucleic acid sequences from one or more samples from one or more locations associated with the one or more unwanted microbes in the environment; compare the one or more identified indicator microbial signatures against one or more databases of indicator microbial signatures that correspond to particular unwanted microbes; and identify one or more unwanted microbes that correspond to one or more of the identified indicator microbial signatures based on the comparison.

In other embodiments, the system may be configured to identify one or more origins of an unwanted microbe in an environment. For example, the system may include one or more processors and a memory comprising instructions executable by the one or more processors. When executed by the one or more processors, the instructions may cause the system to identify one or more origin-indicating microbial signatures within a plurality of nucleic acids sequences obtained from sequencing a plurality of nucleic acid sequences from one or more samples from one or more locations associated with the one or more unwanted microbes in the environment; compare the one or more identified origin-indicating microbial signatures against one or more databases of origin-indicating microbial signatures that correspond to particular origins of unwanted microbes; and identify one or more origins of the one or more unwanted microbes that correspond to one or more of the identified origin-indicating microbial signatures based on the comparison.

In yet other embodiments, the system may be configured to detect an unwanted microbe in a spray drier. For example, the system may include one or more processors and a memory comprising instructions executable by the one or more processors. When executed by the one or more processors, the instructions may cause the system to identify one or more indicator microbial signatures within a plurality of nucleic acid sequences obtained from sequencing a plurality of nucleic acid sequences from one or more samples from one or more locations within the spray dryer, a powder produced by the spray dryer, a liquid to be fed into the spray dryer, or any combination thereof associated with the one or more unwanted microbes in the spray dryer; compare the one or more identified indicator microbial signatures against one or more databases of indicator microbial signatures that correspond to particular unwanted microbes; and identify one or more unwanted microbes that correspond to one or more of the identified indicator microbial signatures based on the comparison.

### VI. Methods in Computer-Readable Storage Devices

Any of the methods described herein can be implemented by computer-executable instructions or code stored in one or more computer-readable medium (e.g., a memory, a magnetic storage, an optical storage, or the like). Such instructions can cause one or more processors to implement the method.

### EXAMPLES

### Example 1

This example describes a microbial source end-to-end trace-back method for detecting unwanted microbes in food product samples and identifying origins thereof in the food product supply chain. The method utilizes microbiome data from raw materials to identify the origins of unwanted microbes by using data from high throughput sequencing of nucleic acids present in the food sample.

FIG. 1 shows an overview of a method for identifying the origin of an unwanted microbe in a food production environment. Samples are collected at any identifiable part of the food manufacturing process in a facility and may include raw materials or finished products of any nature. Nucleic acids (DNA or RNA) are then extracted using commercial kits or using other approaches, such as phenol-chloroform-isoamyl alcohol reagents, Trizol LS, or other reagents that involve the use of guanidium thiocyanate combined with phenol. The extracted nucleic acids are preserved via protective agents such as RNALater, beta-mercaptoethanol, LifeGuard, or equivalent chemical reagents. The nucleic acids are used immediately or stored for later analysis. In the case of RNA samples, the sample is reverse-transcribed prior to further use. The extracted nucleic acids can undergo selective amplification to enrich for specific regions or can undergo genomic scale amplification for further analyses. The nucleic acids may then be amplified at specific regions, or amplified in their entirety using genomic scale amplification, for further analyses. A sequencing library is then prepared by one or more means by commercial kits or a collation of specific reagents to prepare a sequencing library. The sequencing library is then applied to a chip in for sequencing using a high throughput sequencer, such as the Illumina, Oxford Nanopore, ThermoFisher's Ion Torrent, PacBio platforms, or equivalents.

Alternately, the nucleic acids are prepared for microarray applications by shearing and molecular detection reagent labelling for fluorescent, chemiluminescent, or colorimetric detection. Mass spectrometry, capillary gel electrophoresis, and high-performance liquid chromatography can also be used to generate specific patterns of nucleic acid contents. Subsequent detailed analysis of specific molecules, either from the output of the separation technologies, from microarray, or from other solid surface capture techniques, is performed. Any of these methods can be used in combination to generate nucleic acid sequence data, with or without using high throughput sequencing.

The sequence data is then analyzed with the help of bioinformatics by applying several steps, including data quality checking and filtering, creating databases of nucleic acid sequences corresponding to known species, removing any specific parts of the data that are not of interest such as other eukaryotes or specific microbial species, and then identifying an indicator microbial signature or an origin-indicating microbial signature from the sequence data. FIG. 2 depicts an overview of the sequence data analysis process.

The identified microbial members are then traced backwards to the samples from which they were sampled, to help identify their origin in the environment (e.g., in the supply chain, in a location in the factory, in a clinical origin, etc.). Any microbes unique to a specific origin can confirm a specific infection or contamination in the supply chain (FIG. 3). For example, the origin-indicating microbial signature may indicate the origin of a contaminating microbe such as such as Salmonella or E. coli originating from poultry meal or another point in the farm environment, such as farm soil or cow dung tracked on farm worker boots, tracing back to a beef or milk supplier (FIG. 4). Origins of unwanted microbes can also be performed using origin-indicating microbial signatures corresponding to multiple microbial species associated with more than one possible origin (FIG. 5 and FIG. 6). The origin of an unwanted microbe may also be identified without identifying all microbial species present in a food product. Microbial genera associated with particular origins are summarized in Tables 1-5.

TABLE 1
Bacteria signatures at genus taxonomy level unique to various food raw materials.

| Unique to bone | Unique to | | | Unique to corn |
|---|---|---|---|---|
| meal | poultry meal | Unique to fish meal | Unique to egg | powder |
| Parageobacillus | Blautia | Aliivibrio | Porphyrobacter | Shigella |
| Aneurinibacillus | Anaerostipes | Photobacterium | Erythrobacter | Rathayibacter |
| Butyrivibrio | Tyzzerella | Grimontia | Dechloromonas | Leifsonia |
| Coprothermobacter | Intestinimonas | Pseudoalteromonas | Pseudarthrobacter | Arthrobacter |
| Megasphaera | Ethanoligenens | Alteromonas | | Isoptericola |
| Micrococcus | Eubacterium | Colwellia | | Cellulomonas |
| Thermus | Oscillibacter | Yersinia | | Nocardia |
| Meiothermus | Weissella | Edwardsiella | | Gordonia |
| Rahnella | Murdochiella | Oceanimonas | | Propionibacterium |
| Azotobacter | Eggerthella | Marinomonas | | Tessaracoccus |
| Caulobacter | Adlercreutzia | Halomonas | | Pimelobacter |
| Fibrobacter | Gordonibacter | Methylophaga | | Actinoplanes |
| Fervidobacterium | Obesumbacterium | Brucella | | Listeria |
| Methanobrevibacter | Plesiomonas | Caldanaerobacter | | Deinococcus |
| Methanosarcina | Gallibacterium | Synechococcus | | Spirosoma |
| | Thioploca | Calothrix | | |
| | Helicobacter | Thermotoga | | |
| | Janthinobacterium | Nonlabens | | |
| | Barnesiella | Fusobacterium | | |
| | Ornithobacterium | Ilyobacter | | |
| | Akkermansia | Thermodesulfobacterium | | |
| | Cloacibacillus | Theileria | | |
| | Gyrovirus | T7virus | | |
| | T4virus | Alpharetrovirus | | |
| | Sp18virus | | | |

TABLE 2
Bacteria signatures at genus taxonomy level common to any two food raw materials

| Bone | Bone | Bone | Poultry | Corn | Corn | Egg |
|---|---|---|---|---|---|---|
| and Poultry | and Egg | and Fish | and Fish | and Egg | and Fish | and Fish |
| Acidaminococcus | Altererythrobacter | Comamonas | Arcobacter | Aeromicrobium | Pediococcus | Proteus |
| Alistipes | Azospira | Geobacillus | Geoalkalibacter | Agrobacterium | Vibrio | |
| Christensenella | Bosea | Kurthia | Hafnia | Alcaligenes | | |
| Clostridioides | Novosphingobium | Oblitimonas | Morganella | Amycolatopsis | | |
| Odoribacter | | Pseudoxanthomonas | Negativicoccus | Aureimonas | | |
| Olsenella | | Psychrobacter | Paenibacillus | Brachybacterium | | |
| Parabacteroides | | Shewanella | Providencia | Brevibacterium | | |
| Roseburia | | | | Candida | | |
| Ruminococcus | | | | Caulimovirus | | |
| Selenomonas | | | | Clavibacter | | |
| Treponema | | | | Curtobacterium | | |
| Turicibacter | | | | Erwinia | | |
| | | | | Frondihabitans | | |
| | | | | Hymenobacter | | |
| | | | | Kineococcus | | |
| | | | | Kluyveromyces | | |
| | | | | Massilia | | |
| | | | | Methylobacterium | | |
| | | | | Microbacterium | | |
| | | | | Nocardioides | | |
| | | | | Ochrobactrum | | |
| | | | | Pseudonocardia | | |
| | | | | Rhizobium | | |
| | | | | Saccharopolyspora | | |
| | | | | Sanguibacter | | |
| | | | | Shinella | | |
| | | | | Sphingobacterium | | |
| | | | | Sugiyamaella | | |

TABLE 3
Bacteria signatures at genus taxonomy level common to any three food raw materials.

| | | | | Chicken, | |
|---|---|---|---|---|---|
| Bone, Chicken | Bone, Chicken | Bone, Corn | Bone, Egg | Corn and | Corn, Egg and |
| and Corn | and Fish | and Egg | and Fish | Egg | Fish |
| Chryseobacterium | Aeromonas | Achromobacter | Blastomonas | Pantoea | Delftia |
| | Anoxybacillus | Bordetella | | | Mycobacterium |
| | Bacteroides | Brevundimonas | | | Rhodococcus |
| | Bifidobacterium | Kosakonia | | | Streptomyces |
| | Desulfovibrio | Sphingobium | | | Thermothelomyces |
| | Flavonifractor | Sphingomonas | | | Thielavia |
| | Lachnoclostridium | Sphingopyxis | | | |
| | Macrococcus | Cupriavidus | | | |
| | Moraxella | | | | |
| | Prevotella | | | | |
| | Ruminiclostridium | | | | |

TABLE 4
Bacteria signatures at genus taxonomy level common to any four food raw materials.

| Bone, Chicken, | Bone, Chicken, | Bone, Chicken, | Bone, Corn, Egg, | Chicken, Corn, |
|---|---|---|---|---|
| Corn, and Egg | Corn, and Fish | Egg, and Fish | and Fish | Egg, and Fish |
| Bradyrhizobium | Campylobacter | Clostridium | Stenotrophomonas | Burkholderia |
| | Cutibacterium | | Xanthomonas | Serratia |
| | Escherichia | | | Staphylococcus |
| | Streptococcus | | | Variovorax |

TABLE 5
Bacteria signatures at genus taxonomy
level common to all five food raw materials.
Bone, Chicken, Corn, Fish and Egg

Acidovorax
Acinetobacter
Bacillus
Citrobacter
Corynebacterium
Enterobacter
Enterococcus
Klebsiella
Lactobacillus
Lactococcus
Pseudomonas
Raoultella
Salmonella

### Example 2

This example describes the use of metagenomics filtering and indicator microbial signatures and/or origin indicating microbial signatures for identifying microbes and origins thereof in environmental samples.

Identification of microbial signatures from sequence data can be performed as described below, or as described in Beck, K. L., et al. (2021) NPJ Sci Food, 5(1):3. The steps are modified as required by the particular platform employed to obtain the sequence data. Equivalent methods can be used to obtain information on the presence or absence, or of relative levels, of microbial species based on analyses of any DNA or RNA sequences.

### Sample Collection, Preparation, and Sequencing

Animal meal, corn meal, or egg powder samples were collected from a local market in the United States. Sample preparation, total RNA extraction and integrity confirmation, cDNA construction, and library construction for these samples was previously described by Haiminen, N., et al. (2019) NPJ Sci Food 3:24.

The samples were used to extract total RNA as described by Chen, P., et al. (2017) Pathogens 6:68, and total DNA as described elsewhere (Weis, A. M., et al. (2016). Appl. Environ. Microbiol 82:7165-7175; Emond-Rheault, J.-G., et al. (2017) Front. Microbiol. 8:996; Miller, B., et al. (2015) Kapa Biosyst. Appl. Note 1-8 (2015); Lüdeke, C. H. M., et al. (2015) Genome Announc. 3:2-3; Jeannotte, R., et al. (2015) Agil. Appl. Note 1-8; Arabyan, N., et al. (2016) Sci Rep 6: 29525). DNA and RNA purity (A_{260/230} and A_{260/290} ratios ≥1.8) and integrity were confirmed with Nanodrop (Nanodrop Technologies, Wilmington, DE, USA) and BioAnalyzer RNA Kit (Agilent Technologies Inc., Santa Clara, CA, USA) (Chen, P., et al. (2017) Pathogens s6:68). Subsequently, wherever RNA was used, cDNA was constructed using RNA (4 to 15 μg total input) and the SuperScript Double Stranded cDNA Synthesis kit (Invitrogen, Catalog no. 11917-020, Life Technology Carlsbad, CA). DNA processing does not require this particular step

Sequencing libraries using HyperPrep Plus (Kapa BioSystems, Wilmington, MA, USA) cDNA/DNA were constructed as described previously (Chen, P., et al. (2017) Pathogens 6:68; Chen, P., et al. (2017) Appl Env. Microbiol 83; Kol, A., et al. (2014) Stem Cells Dev 23: 1831-1843) with an insert size between 300-400 bp. Library quantification was performed using qPCR (Library Quantification kit, catalog no. KK4824, Illumina, San Diego, CA) prior to submission for sequencing. The Illumina HiSeq 4000 (San Diego, CA) was used with 150 paired-end chemistry for each sample except the following: HiSeq 2000 with 100 paired-end chemistry was used for the four preliminary samples, and HiSeq 3000 with 150 paired-end chemistry was used for two other samples (MFMB-04 and MFMB-17).

### Sequence Data Quality Control

Illumina Universal adapters were removed and reads were trimmed using Trim Galore (Morgulis, A., et al. (2006) J. Comput. Biol. 13:1028-1040) with a minimum read length parameter 50 basepairs (bp). The resulting reads were filtered using Kraken software as described below with a custom database built from the PhiX genome (NCBI Reference Sequence: NC_001422.1). Trimmed non-PhiX reads were used in subsequent matrix filtering and microbial identification steps.

### Matrix Filtering Process and Validation

Kraken (Wood, D. E., and Salzberg, S. L. (2014) Genome Biol. 15:R46), with a k-mer size of 31 bp, was used to identify and remove reads that matched a pre-determined list of 31 common food matrix and potential contaminant eukaryotic genomes. These food matrix organisms were chosen based on preliminary eukaryotic read alignment experiments of the poultry meal samples as well as high-volume food components in the supply chain. Due to the large size of eukaryotic genomes in the custom Kraken database, a random k-mer reduction was applied to reduce the size of the database by 58% (using Kraken-build with option “-max-db-size”), in order to fit the database in 188 GB for in-memory processing. A conservative Kraken score threshold of 0.1 was applied to avoid filtering microbial reads. The matrix-filtering database includes low complexity and repeat regions of eukaryotic genomes to capture all possible matrix reads. This filtering database and the score threshold were also used in the matrix filtering for in silico testing as described below.

### Microbial Identification

Remaining reads after quality control and matrix filtering were classified using Kraken (Wood, D. E., and Salzberg, S. L. (2014) Genome Biol. 15:R46) against a microbial database with a k-mer size of 31 bp to determine the microbial composition within each sample. NCBI RefSeq complete genomes were obtained for bacterial, archaeal, viral, and eukaryotic microorganisms (˜7,800 genomes retrieved in April 2017). Low complexity regions of the genomes were masked using Dustmasker (Morgulis, A., et al. (2006) J. Comput. Biol. 13:1028-1040) with default parameters. A threshold of 0.05 was applied to the Kraken score in an effort to maximize the F-score of the result. Taxa-specific sequence reads were used to identify presence or absence of microbial species, with a minimum of 10 reads required as the threshold for positive presence determination.

### Other Potential Mechanisms of Filtering the Data for Isolating Microbial Signatures

Apart from using DNA or RNA sequences, the nucleic acid sequences can also be 6-frame-translated to protein sequences prior to comparison against databases for filtering out eukaryotic signatures. This can be a more efficient way to manage the data volume. This can be performed using any DNA or protein sequence matching or alignment tool known to experts such as BLAST, bowtie2, bwa, MUMmer, Mash, MUSCLE, T-Coffee, or equivalent tools. These tools may either use direct alignments or kmer hashing for identifying best matches.

### Microbial Origin Tracking

This is the process of using the isolated microbial sequences and comparison at various levels to create dendrograms or phylogenetic trees for tracking back to the origin. This can be performed specifically within the isolated origin-identifying microbial signatures across various samples conceivably using any DNA, RNA, or protein sequence matching or alignment tool known to experts such as BLAST, bowtie2, bwa, MUMmer, Mash, MUSCLE, T-Coffee, or equivalent tools. These tools may either use direct alignments or kmer hashing for identifying genomic distances between the samples. This can be based on identifying kmer hash-based or overall alignment level based genomic distances, such as described by Ondov et al. or Meier-Kolthof et al. or identifying SNPs using various tools such as MUMmer, Parsnp, the CFSAN SNP pipeline or other novel tools.

### Example 3

This example describes how the methods described in Examples 1 and 2 can be used for detecting unwanted microbes and origins thereof in a variety of applications in the food production and public health sectors (FIG. 7).

### Metagenomic Data Filtering for Food Quality and Safety and Surrounding Environmental Safety

In this specific use case, at any point of the supply chain form farm to fork, such as even agricultural or farm soil, transport vehicle surfaces, factory material reception equipment, factory floors, food processing material and equipment, and finished product may be sampled to determine microbial composition and to identify all possible microbes found within the collected sample. The presence of common microbes across the samples is used in the traceback process to determine whether or not the product is of acceptable quality.

### Metagenomic Data Filtering for Pathogen Determination Applicable to Generic Product Manufacturing Lines Including Testing Food and Related Materials' Transport Vehicles and Operators for Pathogenic Transmission and Carryover

Here, food processing material and equipment and finished product may be sampled to determine microbial composition and to identify specific pathogens found within the collected sample. The difference from the prior case is that specific pathogens can be subjected to targeted amplification prior to high throughput sequencing. This allows increased sensitivity of pathogen testing. Again, the presence/absence of pathogens determines the safety of the product and/or facility being surveyed.

### Metagenomic Data Filtering for Testing Office Facility/Public Restrooms for Pathogenic Transmission and Carryover

Public and private restrooms may be sampled to determine microbial composition and to identify specific pathogens found within the collected sample. Specific pathogens may or may not be subjected to targeted amplification prior to high throughput sequencing. Prior PCR amplification allows increased sensitivity of pathogen testing. The presence/absence of pathogens determines the safety of the facility being surveyed.

Metagenomic Data Filtering for Assessing Quality Safety of Food Powder Spray Driers with Product Caking

This is a special application of metagenomics to dairy powders to understand their microbiome as a measure of spray drier performance and aid in cleaning practices. Food products are spray dried in some facilities. The inlet is a concentrated liquid food from which most of the water content is evaporated during the spray drying process to obtain a powdered product form. Such products still contain low levels of viable bacteria or bacterial spores, especially in protein rich products such as dairy milks. These bacteria or spores are known to form components of biofilms and aid in product caking at inlets. Understandably, the levels of these bacteria or spores are higher in product lumps, biofilms, or areas of the drier where the product may form a thick layer or caking. Using metagenomics to assess the levels of such bacteria or spores allows us to determine quality and additionally manage the cleaning process suitably. For example, when only low levels of such bacteria or spores are detected, a different cleaning regime may be preferred vs when the caking is richer in bacteria.

### Metagenomic Data Filtering for any Bacterial and or Pathogenic Transmission and Carryover In Animal Transport Vehicles

Here, vehicles used in pet or other animal transport may be sampled to determine microbial composition and to identify specific pathogens found within the collected sample. Transport vehicles may be a source of zoonotic infectious diseases. Either specific pathogens can be subjected to targeted amplification prior to high throughput sequencing or whole swabs or fecal samples or any type of related surface/material samples may be collected to test and identify unwanted bacterial species. The amplification approach allows increased sensitivity of pathogen testing. The presence/absence of pathogens and traceback process determines the safety of the vehicles being surveyed and decisions of whether the vehicle is safe for transport.

1. A method for detecting one or more unwanted microbes in an environment, the method comprising:
obtaining one or more samples from one or more locations associated with the one or more unwanted microbes in the environment;
sequencing a plurality of nucleic acid sequences within the one or more samples;
identifying one or more indicator microbial signatures within the plurality of nucleic acid sequences;
comparing the one or more identified indicator microbial signatures against one or more databases of indicator microbial signatures that correspond to particular unwanted microbes; and
identifying one or more unwanted microbes that correspond to one or more of the identified indicator microbial signatures based on the comparison.

2. The method of claim 1, wherein the one or more indicator microbial signatures comprise nucleic acid sequences from microbes other than the one or more unwanted microbes and/or do not comprise nucleic acid sequences from the one or more unwanted microbes.

3. (canceled)

4. A method for identifying one or more origins of one or more unwanted microbes in an environment, the method comprising:
obtaining one or more samples from one or more locations associated with the one or more unwanted microbes in the environment;
sequencing a plurality of nucleic acid sequences within the one or more samples;
identifying one or more origin-indicating microbial signatures within the plurality of nucleic acids sequences;
comparing the one or more identified origin-indicating microbial signatures against one or more databases of origin-indicating microbial signatures that correspond to particular origins of unwanted microbes; and
identifying one or more origins of the one or more unwanted microbes that correspond to one or more of the identified origin-indicating microbial signatures based on the comparison.

5-22. (canceled)

23. The method of claim 1, wherein the environment is a food production environment.

24. The method of claim 23, wherein the food production environment is selected from the group consisting of a farm, a food transport vehicle, an animal transport vehicle, a food processing facility, a food packaging facility, a food distribution facility, a warehouse, and a food market.

25-26. (canceled)

27. The method of claim 1, wherein the one or more samples comprise samples from agricultural or farm soil, agricultural or farm equipment, crops, livestock, transport vehicle surfaces, factory equipment, factory floors, food processing material, food processing equipment, food products, worker apparel, or any combination thereof.

28. The method of claim 1, wherein the environment is an environment in a clinic, public building, an office building, a residential building, or an animal care facility.

29. The method of claim 28, wherein the one or more samples comprise samples from a companion animal, a service animal, an item or surface contacted by a companion animal or a service animal, or any combination thereof.

30. (canceled)

31. The method of claim 1, wherein the environment is a spray dryer.

32. A method for detecting one or more unwanted microbes in a spray dryer, the method comprising:
obtaining one or more samples from one or more locations within the spray dryer, from a powder produced by the spray dryer, or from a liquid to be fed into the spray dryer, or any combination thereof in the spray dryer, around the spray dryer, or both;
sequencing a plurality of nucleic acid sequences within the one or more samples;
identifying one or more indicator microbial signatures within the plurality of nucleic acid sequences;
comparing the one or more identified indicator microbial signatures against one or more databases of indicator microbial signatures that correspond to particular unwanted microbes; and
identifying one or more unwanted microbes that correspond to one or more of the identified indicator microbial signatures based on the comparison.

33-38. (canceled)

39. The method of claim 1, wherein the one or more indicator microbial signatures correspond to the genus taxonomy, the species taxonomy, the serotype taxonomy, or the relative abundance of one or more unwanted microbes present in the environment.

40-41. (canceled)

42. The method of claim 1, wherein the one or more unwanted microbes are selected from the group consisting of bacteria, viruses, archaea, and eukaryotic microorganisms.

43. The method of claim 1, wherein the one or more unwanted microbes belong to a genus taxonomy selected from the group consisting of: Parageobacillus, Blautia, Aliivibrio, Porphyrobacter, Shigella, Aneurinibacillus, Anaerostipes, Photobacterium, Erythrobacter, Rathayibacter, Butyrivibrio, Tyzzerella, Grimontia, Dechloromonas, Leifsonia, Coprothermobacter, Intestinimonas, Pseudoalteromonas, Pseudarthrobacter, Arthrobacter, Megasphaera, Ethanoligenens, Alteromonas, Isoptericola, Micrococcus, Eubacterium, Colwellia, Cellulomonas, Thermus, Oscillibacter, Yersinia, Nocardia, Meiothermus, Weissella, Edwardsiella, Gordonia, Rahnella, Murdochiella, Oceanimonas, Propionibacterium, Azotobacter, Eggerthella, Marinomonas, Tessaracoccus, Caulobacter, Adlercreutzia, Halomonas, Pimelobacter, Fibrobacter, Gordonibacter, Methylophaga, Actinoplanes, Fervidobacterium, Obesumbacterium, Brucella, Listeria, Methanobrevibacter, Plesiomonas, Caldanaerobacter, Deinococcus, Methanosarcina, Gallibacterium, Synechococcus, Spirosoma, Thioploca, Calothrix, Helicobacter, Thermotoga, Janthinobacterium, Nonlabens, Barnesiella, Fusobacterium, Ornithobacterium, Ilyobacter, Akkermansia, Thermodesulfobacterium, Cloacibacillus, Theileria, Gyrovirus, T7virus, T4virus, Alpharetrovirus, Sp18virus, Acidaminococcus, Altererythrobacter, Comamonas, Arcobacter, Aeromicrobium, Pediococcus, Proteus, Alistipes, Azospira, Geobacillus, Geoalkalibacter, Agrobacterium, Vibrio, Christensenella, Bosea, Kurthia, Hafnia, Alcaligenes, Clostridioides, Novosphingobium, Oblitimonas, Morganella, Amycolatopsis, Odoribacter, Pseudoxanthomonas, Negativicoccus, Aureimonas, Olsenella, Psychrobacter, Paenibacillus, Brachybacterium, Parabacteroides, Shewanella, Providencia, Brevibacterium, Roseburia, Candida, Ruminococcus, Caulimovirus, Selenomonas, Clavibacter, Treponema, Curtobacterium, Turicibacter, Erwinia, Frondihabitans, Hymenobacter, Kineococcus, Kluyveromyces, Massilia, Methylobacterium, Microbacterium, Nocardioides, Ochrobactrum, Pseudonocardia, Rhizobium, Saccharopolyspora, Sanguibacter, Shinella, Sphingobacterium, Sugiyamaella, Chryseobacterium, Aeromonas, Achromobacter, Blastomonas, Pantoea, Delftia, Anoxybacillus, Bordetella, Mycobacterium, Bacteroides, Brevundimonas, Rhodococcus, Bifidobacterium, Kosakonia, Streptomyces, Desulfovibrio, Sphingobium, Thermothelomyces, Flavonfractor, Sphingomonas, Thielavia, Lachnoclostridium, Sphingopyxis, Macrococcus, Cupriavidus, Moraxella, Prevotella, Ruminiclostridium, Bradyrhizobium, Campylobacter, Clostridium, Stenotrophomonas, Burkholderia, Cutibacterium, Xanthomonas, Serratia, Escherichia, Staphylococcus, Streptococcus, Variovorax, Acidovorax, Acinetobacter, Bacillus, Citrobacter, Corynebacterium, Enterobacter, Enterococcus, Klebsiella, Lactobacillus, Lactococcus, Pseudomonas, Raoultella, and Salmonella.

44. The method of claim 1, wherein sequencing the plurality of nucleic acid sequences within the one or more samples comprises preparing a sequencing library, next generation sequencing, microarray analysis, or any combination thereof.

45. (canceled)

46. The method of claim 1, wherein the plurality of nucleic acid sequences comprise DNA sequences, RNA sequences, or a combination thereof.

47. (canceled)

48. The method of claim 1, wherein non-microbial sequences are filtered from the plurality of nucleic acid sequences prior to identifying the one or more indicator microbial signatures.

49. The method of claim 1, wherein the one or more databases are databases of microbial nucleic acid sequences.

50. A system for carrying out the method of claim 1, comprising:
one or more processors; and
a memory comprising instructions executable by the one or more processors that, when executed by the one or more processors, cause the system to:
identify one or more indicator microbial signatures within a plurality of nucleic acid sequences obtained from sequencing a plurality of nucleic acid sequences from one or more samples from one or more locations associated with the one or more unwanted microbes in the environment;
compare the one or more identified indicator microbial signatures against one or more databases of indicator microbial signatures that correspond to particular unwanted microbes; and
identify one or more unwanted microbes that correspond to one or more of the identified indicator microbial signatures based on the comparison.

51. A system for carrying out the method of claim 4, comprising:
one or more processors; and
a memory comprising instructions executable by the one or more processors that, when executed by the one or more processors, cause the system to:
identify one or more origin-indicating microbial signatures within a plurality of nucleic acids sequences obtained from sequencing a plurality of nucleic acid sequences from one or more samples from one or more locations associated with the one or more unwanted microbes in the environment;
compare the one or more identified origin-indicating microbial signatures against one or more databases of origin-indicating microbial signatures that correspond to particular origins of unwanted microbes; and
identify one or more origins of the one or more unwanted microbes that correspond to one or more of the identified origin-indicating microbial signatures based on the comparison.

52. A system for carrying out the method of claim 32, comprising:
one or more processors; and
a memory comprising instructions executable by the one or more processors that, when executed by the one or more processors, cause the system to:
identify one or more indicator microbial signatures within a plurality of nucleic acid sequences obtained from sequencing a plurality of nucleic acid sequences from one or more samples from one or more locations within the spray dryer, a powder produced by the spray dryer, a liquid to be fed into the spray dryer, or any combination thereof associated with the one or more unwanted microbes in the spray dryer;
compare the one or more identified indicator microbial signatures against one or more databases of indicator microbial signatures that correspond to particular unwanted microbes; and
identify one or more unwanted microbes that correspond to one or more of the identified indicator microbial signatures based on the comparison.

53-54. (canceled)
